(19) United States
(12) Patent Application Publication
(10) Pub. No.: US 2026/0060734 A1
(43) Pub. Date: Mar. 5, 2026

(54) METHOD AND SYSTEM FOR IRREVERSIBLE ELECTROPORATION

(71) Applicant: Cultiv8 Medical, LLC, Minneapolis, MN (US)

(72) Inventors: J. Samuel BATCHELDER, Minneapolis, MN (US); Matt HEIDNER, Minneapolis, MN (US); Jon LAWSON, Minneapolis, MN (US)

(73) Assignee: Cultiv8 Medical, LLC, Minneapolis, MN (US)

(21) Appl. No.: 19/307,840

(22) Filed: Aug. 22, 2025

Related U.S. Application Data
(60) Provisional application No. 63/688,169, filed on Aug. 28, 2024.

(51) Int. Cl. A61B 18/00

(52) U.S. Cl. CPC A61B 18/00; A61B 2018/00613; A61B 2018/00702; A61B 2018/00714; A61B 2018/0091

(57) ABSTRACT

A system for performing irreversible electroporation within a tissue under a skin surface of a patient, the system including: a power source configured to generate power; a base; and at least one electrode extending from the base, where the at least one electrode is configured to receive the power and based on the power to generate an electric field sufficient to porate a cell membrane within the tissue while increasing a temperature of the skin surface less than 3 deg C. during poration.

### CROSS-REFERENCE TO RELATED APPLICATION(S)

This application claims the benefit of U.S. Provisional Application No. 63/688,169, filed Aug. 28, 2024, the entire disclosure of which is incorporated herein by reference.

### FIELD OF THE INVENTION

The disclosure relates to a method and a system for irreversible electroporation (IRE), as well as associated methods and systems for treatment of skin conditions.

### BACKGROUND

Irreversible electroporation (IRE) may be used for the treatment of various conditions or diseases, including certain types of cancer. During IRE treatment, a voltage, such as in time-varying pulse trains, may be provided to one or more electrodes, which generate an external electric field, and the external electric field may be applied to various organs or other parts of the body of a patient. The electric field may cause pores to form in cells or vesicle membranes when the external electric field induces about a one volt potential difference across the cells or membranes. The pores in the treated cells may cause the cells or membranes to undergo cell death or lysis either immediately or over time. For a typical 10 nm thick membrane, this potential difference may require an electric field of about 10⁶ volts/cm, which may be sufficient to break down lipid layers in the cells or membranes. This rule changes with non-round long cells and field orientations, and may be off by a factor of 2, for example.

Although IRE treatments generally result in an increase in temperature in the treated organs or other body parts, an IRE treatment may be considered to be non-thermal when the temperature change does not produce any undesired side effects. Depending on the bodily organs involved in the IRE treatment, temperature changes between about 0.1 deg C. and about 4 deg C. for short time periods may be considered non-thermal. However, a tip of a needle used during penetrating IRE treatments may generate electric fields of greater than 100 KV/cm at the needle surface, even at moderate drive voltages, causing thermal damage at locations near the needle. Furthermore, current needle IRE treatments may not apply uniform electric field strengths over 10 KV/cm throughout the entire volume of the targeted organ or body part undergoing treatment.

Known IRE treatments may not apply 10⁶ volts/cm to the entire volume of the organ or other body part undergoing treatment, in part due to destructive heating of the samples. Such a field would dissipate ˜7 gigawatt/cc in saline, with the temperature rising at roughly 2 deg C./nsec. Instead, most IRE treatments rely on the phenomenon that ionic conduction, within the electrolyte that permeates biological samples, may tend to accumulate on insulating interfaces, such as on the membranes. The accumulated ions may act to cancel the field in the electrolyte while amplifying the field in the membranes

Although a relatively strong electric field may porate cell membranes with a single 30 ns pulse, for example, known IRE treatments may apply tens to thousands of relatively weaker pulses. The nth pulse may porate a particular cell although the previous (n−1) pulses may not.

Additionally, electrical conductivity throughout the tissue that is undergoing IRE treatment may not be homogenous. For example, an isolated vesicle in an electrolyte may experience a potential difference over its length d_{cell} in the direction of the electric field E which is initially about Eₑₓₜ d_{cell}. When the material in the vesicle is also an electrolyte, ions may drift to the membrane surface, which as stated may cancel the external electric field, creating a potential drop across the membrane at diametrically opposite ends of the cell of about Eₑₓₜ d_{cell}/2. Further, relatively large vesicles may porate at lower field strengths than relatively small cells. Still further, once a membrane has several pores, ionic conduction may pass through the vesicle nearly as well as if the vesicle were a bulk electrolyte.

Moreover, cells may differ from one another, and individual cells may have internal organelles that facilitate or impede ion mobility within the cells. Further, a cell that is close-packed by other non-porated cells may experience a lower ionic mobility external to that cell. Thus, when the local conductivity for ionic conduction is low, the ions may not drift quickly to the membrane of the cell, and poration may not occur. When multiple poration pulses are applied to the tissue, portions of the cells in the tissue or membrane which are better conductors may porate, may change their average conductivity, which may change the external electrical field perceived by other neighboring cells. Given enough pulses, all of the cells in the sample larger than d_{cell}=2V/Eₑₓₜ may porate. The presence of smaller cells or thicker membranes may result in the ionic current traversing a more tortuous paths, which may slow down pore formation in the tissue or membrane.

Moving electrodes over the surface of biological tissue has advantages over prior art electrodes that are inserted or placed stationary on tissue: 1) the IRE dose can be made more uniform because of the averaging effect of motion; 2) the treatment depth can be designed by the geometry of the electrodes, the form of the electrical pulses, and the pulse density during motion; and 3) non-thermal IRE treatment can be insured by eliminating local high-field regions in the electrode mounting; this is done by enforcing a minimum surface radius on the electrodes, and by grouting the electrodes with semiconducting material where insulating support, electrode surface, and tissue are in close contact. In particular, near-surface fields of approximately 50 KV/cm can sterilize wound near-surface bacteria. An embodiment of a scanning surface IRE apparatus is a self-contained portable field applicator that is manually scanned over the tissue area of interest.

The disclosed methods and systems may overcome one or more of the above discussed disadvantages, or other disadvantages, which are known or unknown in the art.

### SUMMARY OF CLAIMED SUBJECT MATTER

In accordance with some aspects of the disclosure, a system for performing irreversible electroporation within a tissue under a skin surface of a patient comprises: a power source configured to generate power; a base; and at least one electrode extending from the base, wherein the at least one electrode is configured to receive the power and based on the power to generate an electric field sufficient to porate a cell membrane within the tissue while increasing a temperature of the skin surface less than 3 deg C. during poration.

In accordance with other aspects of the disclosure, a hand-held applicator for performing irreversible electroporation within a tissue under a skin surface of a patient, comprises: a base; and at least one electrode extending from the base, wherein the at least one electrode is configured to generate an electric field having a maximum field strength equal to a potential difference applied to the tissue divided by a radius of curvature of a surface of the at least one electrode, while providing a treatment field of less than 0.3 times the maximum field strength a distance of 1 mm from the at least one electrode

In accordance with still other aspects of the disclosure, method of performing irreversible electroporation within a tissue under a skin surface of a patient, comprises: applying power to at least one electrode in contact with the skin surface of the patient; generating with the at least one electrode, based on the applied power, an electric field, wherein the electric field is sufficient to porate a cell membrane within the tissue while increasing a temperature of the skin surface less than 3 deg C. during poration; and applying the electric field to porate the cell membrane in the tissue

### BRIEF DESCRIPTION OF THE FIGURES

The accompanying drawings, which are incorporated in and constitute a part of this specification, illustrate various exemplary embodiments, and together with the description, serve to explain principles of the disclosed embodiments.

FIG. 1 is a table illustrating three different IRE treatments protocols, in accordance with aspects of the disclosure.

FIG. 2 is a graph illustrating data from FIG. 1, the graph showing electric field strength, cell size, peak energy in Watts, and pulses per treatment, for three tissue sample volumes, in accordance with aspects of the disclosure.

FIG. 3 is a graph illustrating an electric field versus distance from the electrode during IRE treatment, in accordance with aspects of the disclosure.

FIG. 4 is a graph illustrating electric field strength versus cell size, in accordance with aspects of the disclosure.

FIG. 5 is a depiction of an output of a finite element analysis model of an electrode applying an electric field during IRE treatment, illustrating data from FIG. 4, in accordance with aspects of the disclosure.

FIG. 6 is a graph illustrating temperature rise of tissue undergoing IRE treatment versus distance from an electrode, in accordance with aspects of the disclosure.

FIG. 7 illustrates a schematic view of an IRE treatment system, in accordance with aspects of the disclosure.

FIG. 8 illustrates a bottom isometric view of an applicator for IRE treatment, in accordance with aspects of the disclosure.

FIG. 9 illustrates a detail view of portions of the applicator of FIG. 8 against the skin of a patient, in accordance with aspects of the disclosure.

FIG. 10 illustrates a detail view of a base including electrodes of the applicator of FIG. 8, in accordance with aspects of the disclosure.

FIG. 11 illustrates schematic diagrams showing different configurations of electric fields emerging from a spherical or cylindrical electrode, in accordance with aspects of the disclosure.

FIG. 12 illustrates a further detail view of a portion of the applicator of FIG. 8 against the skin of the patient, in accordance with aspects of the disclosure.

FIG. 13 illustrates a side view of a portion of the applicator of FIG. 8, in accordance with aspects of the disclosure.

FIG. 14 illustrates another side view of a portion of the applicator of FIG. 8, in accordance with aspects of the disclosure.

FIG. 15 illustrates a bottom isometric view of the applicator of FIG. 8, in accordance with aspects of the disclosure.

FIG. 16 illustrates another side view of a portion of the applicator of FIG. 8 in a flexed configuration, in accordance with aspects of the disclosure.

FIG. 17 illustrates another side view of a portion of the applicator of FIG. 8 in another flexed configuration, in accordance with aspects of the disclosure.

FIG. 18 illustrates a bottom isometric view of another embodiment of an applicator, in accordance with aspects of the disclosure.

FIG. 19 illustrates a detail view of the applicator of FIG. 18, in accordance with aspects of the disclosure.

FIG. 20 illustrates a bottom isometric view of another embodiment of an applicator, in accordance with aspects of the disclosure.

FIG. 21 illustrates a cross-sectional view of a portion of the applicator of FIG. 20 applying an electric field to the skin of a patient, in accordance with aspects of the disclosure.

FIG. 22 illustrates a top isometric view of a portion of another embodiment of an applicator, in accordance with aspects of the disclosure.

FIG. 23 illustrates a side cross-sectional view of the applicator of FIG. 22 applying an electric field to the skin of a patient, in accordance with aspects of the disclosure.

FIG. 24 illustrates a schematic isometric view of the three-dimensional electric field, in accordance with aspects of the disclosure.

FIG. 25 illustrates a partial cross-sectional view of another embodiments of an applicator, in accordance with aspects of the disclosure.

FIGS. 26-30 illustrate views of another embodiment of an applicator, in accordance with aspects of the disclosure.

FIG. 31 is a graph illustrating electromagnetic field strength versus a distance from an electrode in volts per centimeter, and a graph illustrating temperature rise versus the distance from the electrode, in accordance with aspects of the disclosure.

FIG. 32 is a graph illustrating temperature versus contact time measured at an interface between an electrode and the skin, based on animal testing, in accordance with aspects of the disclosure.

FIG. 33 is a graph illustrating impedance versus pulse count comparison of high and low voltages, in accordance with aspects of the disclosure.

FIG. 34 is a schematic illustrating a generator, in accordance with aspects of the disclosure.

FIG. 35 is a graph illustrating characteristics of bipolar pulse, in accordance with aspects of the disclosure.

FIG. 36 shows a Vvedensky pulse former for IRE treatment, in accordance with aspects of the disclosure.

FIG. 37 is a graph depicting electromagnetic field strength versus a distance from an electrode in volts per centimeter, as well as a graph of an associated temperature rise versus the distance from the electrode, in accordance with aspects of the disclosure.

FIG. 38 illustrates a finite element analysis model of electrodes on tissue, in accordance with aspects of the disclosure.

FIG. 39 illustrates cells in the dermal layer, in accordance with aspects of the disclosure.

FIG. 40 illustrates histology from an animal experiment, in accordance with aspects of the disclosure.

FIGS. 41-43 illustrate surface location field treatment from an applicator, in accordance with aspects of the disclosure.

### DETAILED DESCRIPTION

Both the foregoing general description and the following detailed description are exemplary and explanatory only, and are not restrictive of the various features as claimed. As used herein, the terms “comprises,” “comprising,” “having,” “including,” or other variations thereof are intended to cover a non-exclusive inclusion such that a process, method, article, or apparatus that comprises, has, or includes a list of elements does not necessarily include only those elements, but may include other elements not expressly listed or inherent to such a process, method, article, or apparatus. Moreover, in this disclosure, relative terms, such as, for example, “about,” “substantially,” “generally,” and “approximately” may indicate a possible variation of ±10% in the stated value.

In accordance with aspects of the disclosure, the shown and described methods and systems may provide IRE (irreversible extraporation) treatment, in which an electrode moves across the skin of a patient for example, such as to treat the skin or the tissue of the patient, as set forth in more detail below. In accordance with the terms used in this disclosure irreversible electroporation IRE/pulsed electric field PEF/and pulsed field ablation PFA, all describe the application of electric field to tissue to cause cellular changes or effects.

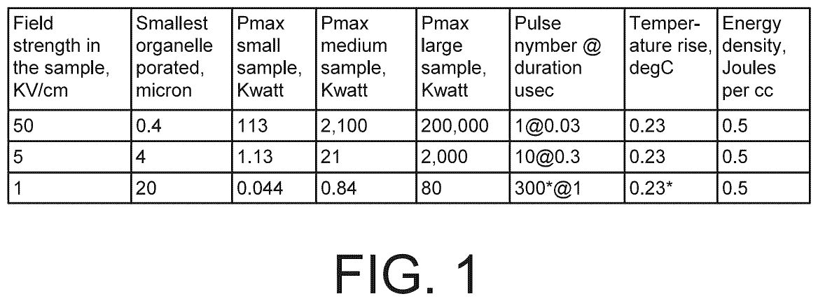

FIG. 1 is a table disclosing three different IRE treatments protocols, in accordance with aspects of the disclosure. The table of FIG. 1 describes the development of nine examples of IRE treatment as a matrix of three cell or organelle sizes, and three tissue areas and depths. The smallest organelle or cell size to be treated determines the minimum field strength required, so that an additional 1 volt may develop across the organelle membrane at either end of the cell in the direction of the field. At one end of the spectrum 0.4 microns represent small bacteria, and at the opposite end, 20 microns represents muscle cells.

A small sample to be treated with each pulse is assumed to be 0.3 mm in depth, with an area of 0.5 mm×12 mm under each of the cylindrical electrodes in the manual scanner using shallow (small diameter) electrodes. A medium sample to be treated with each pulse is assumed to be a 2 mm in depth with an area of 2 mm×10 mm under each of the cylindrical electrodes in a manual scanner using deep (large dimeter) electrodes. A large sample to be treated with each pulse is assumed to be 10 mm in depth with an area of 30 mm×40 mm under an interdigitated electrode array applied to the skin as a stationary patch. The 50 KV/cm electric fields generally achieve treatment in a single 30 ns pulse for all of the organelle sizes 0.4 microns and up. As the field is reduced, more duration is required due to several effects, including current barriers composed of other organelles too small to porate, parallel current paths that divert current from charging the membranes of the target organelles, and increased ion drift time required for the longer organelle dimensions. When the field is as low as 1 KV/cm, it has been shown that on the order of 300 pulses of 1 us duration are required, due to the attenuation effects just described. Temperature rise of the tissue is due to the field strength squared times the conductivity. The temperature rises shown assume the electrical conductivity of saline; for most tissue the temperature rise will be somewhat less. For typical tissue, the heat capacity is about 2 J/(deg C.*cc), giving the displayed energy density deposited in the treated tissue. The asterisk in FIG. 1 indicates that the energy density deposited by the accumulation of 300 pulses of 1 usec length at 1 KV/cm is about 2.1 J/cc, corresponding to a 1 deg C. rise. The listed 0.23 deg C. rise assumes that the pulses are applied over several seconds, allowing time for the accumulated heat to be conducted and convected from the treated tissue.

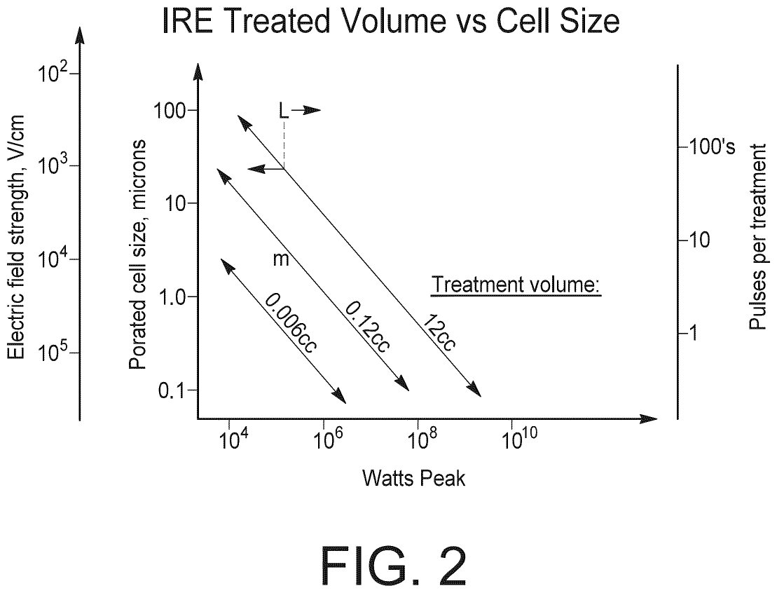

FIG. 2 is a graph illustrating data from FIG. 1, the graph indicating electric field strength, cell size, energy in Watts, and pulses per treatment, for three tissue sample volumes, in accordance with aspects of the disclosure. As FIG. 2 illustrates, power requirements for IRE treatments may depend on the sample volume. An example of a sample with a small volume is a sample with an area of 0.5 mm×12 mm under one or more electrodes that treat the sample to a depth of about 0.3 mm, for a volume of 0.006 cc. An example of a sample with a medium volume is a sample with an area of 2 mm×10 mm under one or more electrodes that treat the sample to a depth of about 2 mm, for a volume of 0.12 cc. An example of a sample with a large volume is a sample with an area of 30 mm×40 mm under one or more electrodes that treat the sample to a depth of a 10 mm depth, for a volume of 12 cc. The energy used to treat the small, medium, and large sample volumes may be about 0.005 J, 0.06 J, and 6 J, respectively. As shown in FIG. 2, treating relatively small cells or organelles generally may require high peak power.

As further described below, the systems for IRE treatment may include one or more electrodes that generate an external electric field, and apply the external electric field to the tissue undergoing treatment. The electric field in a dielectric material (e.g., tissue) immediately adjacent to an electrode which is in the form of a conductive cylinder may be calculated analytically, in accordance with the following discussion. For example, the dielectric material may have a permittivity ε. The cylindrical electrode may have a radius rₑ, and may carry an excess charge per unit length λ. In such cases, a resulting radial electric field in the tissue immediately adjacent to the electrode may be expressed by the following equation:

$$\overline{E} = \frac{λ}{2 π {ε}_{0} ε r} \hat{r}$$

Assume that the maximum field strength is twice the lethal electroporation threshold (LET); Ēₘₐₓ=2 Ēₜₕᵣₑₛₕ. Because

$${\overline{E}}_{max} = \frac{λ}{2 π {ε}_{0} ε {r}_{e}} \hat{r} ,$$

then the radius of the treatment at Ēₜₕᵣₑₛₕ is r=2rₑ, making the effective range of treatment from the surface rₑ. According to the equations, a treatment depth of 0.3 mm therefore may be provided by a cylindrical electrode with a 0.6 mm diameter, which may be suitable for treatment of various tissue types and diseases, including skin diseases.

When the range of the electric field is smaller than a diameter of a typical skin cell, there may be a natural resistance of the skin cells to electroporation. For example, when the radius of the electrode contacting the skin is 30 microns or less, the electric field from the electrode may fall below an electroporation threshold at or near a far side of the cell (e.g., a side of the cell distal from the electrode).

When a second cylindrical electrode applies an external electric field to the skin, the second electrode parallel to the first to the first electrode with a distance 2dₑ between the axes of the two electrodes, then the electric field may be expressed by the following equation:

$$\vec{E} ( x , y , t ) = \frac{V ( t ) \sqrt{{d}_{e}^{2} − {r}_{e}^{2}} \{ 2 x y {\hat{e}}_{x} + ( {y}^{2} − {x}^{2} − {d}_{e l}^{2} + {r}_{e}^{2} ) {\hat{e}}_{y} \}}{( {( y + \sqrt{{d}_{e}^{2} − {r}_{e}^{2}} )}^{2} + {x}^{2} ) ( {( y − \sqrt{{d}_{e}^{2} − {r}_{e}^{2}} )}^{2} + {x}^{2} ) ln ( \frac{{d}_{e}}{{r}_{e}} + \sqrt{\frac{{d}_{e}^{2}}{{r}_{e}^{2}} − 1} )}$$

When the two electrodes are far apart, e.g., when dₑ>>rₑ, a voltage difference between the two cylinders may be expressed by the following equation:

$${V}_{diff} = {r}_{e} ln ( \frac{2 {d}_{e}}{{r}_{e}} ) {E}_{max}$$

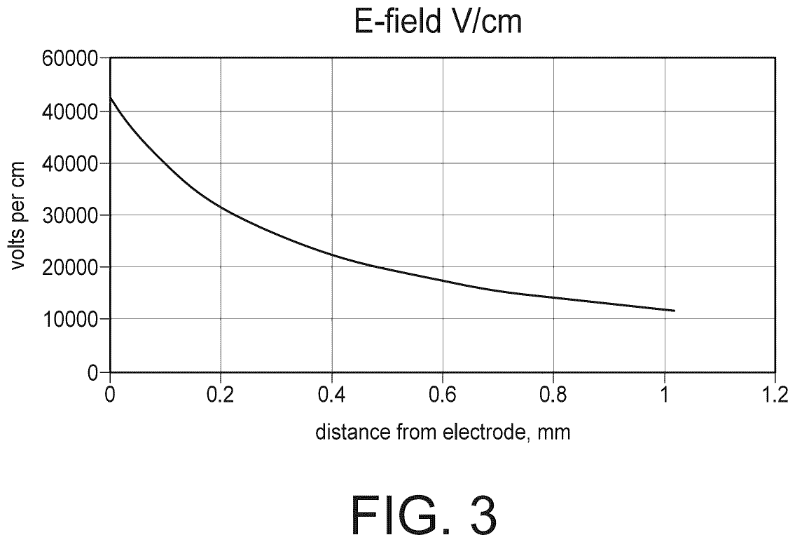

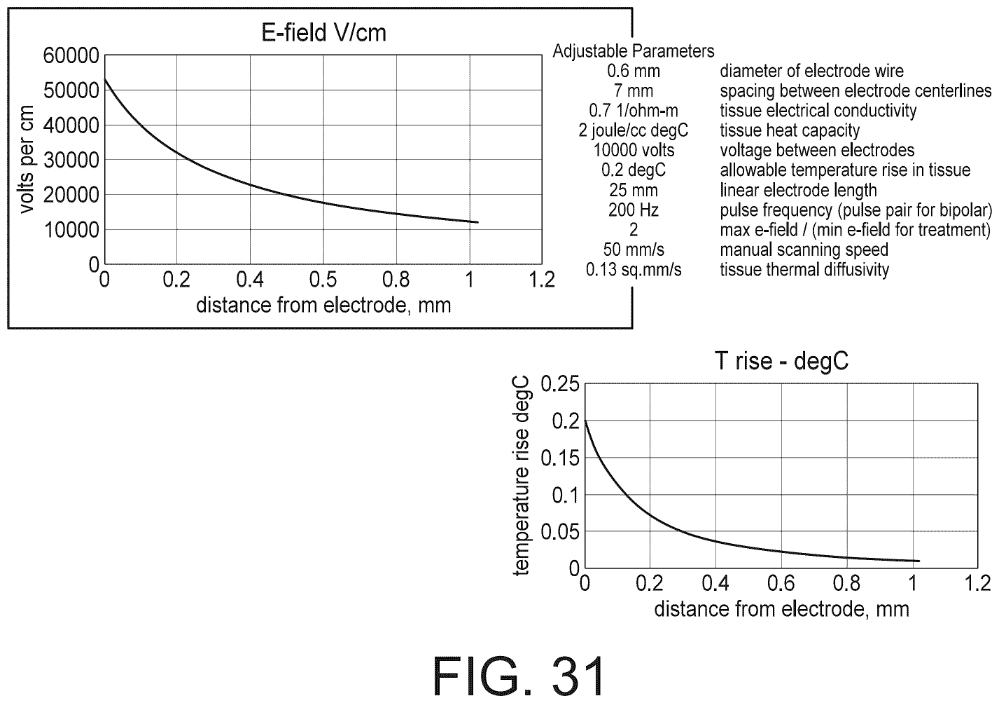

This equation indicates that the electric field intensity may be relatively high when the electrode radius is relatively small, without resulting in arcing between the electrodes, or without requiring higher supply voltages during the IRE treatment. For example, a porating field Eₘₐₓ=50 KV/cm for the electrode having a diameter of 0.6 mm when there is a second electrode spaced 50 mm away from the first electrode, in accordance with the disclosure, may require a voltage difference between the electrodes of only 8.7 KV, corresponding to an air arc distance of 3 mm. In contrast, a parallel plate capacitor with the same 50 mm separation may require 250 KV to generate 50 KV/cm between the electrodes. Thus, treatment a device that includes the 0.6 mm cylindrical electrodes may provide a 29× field amplification as compared to the parallel plate capacitor. This relationship between the electric field provided to the cells and the distance from the electrode is illustrated in the graph of FIGS. 3 and 31.

A single cylindrical electrode with an excess charge per unit length λ_{cyl} a diameter d_{cyl} and immersed in a dielectric of permittivity ε, may produce a local radial electric field represented by the following equation:

$${\overline{E}}_{cyl} = \frac{{λ}_{cyl}}{2 π {ε}_{0} ε r} \hat{r}$$

Two wires each with diameter d_{cyl} formed into two concentric ring-shaped electrodes, with respective centerline diameters Dₒ and Dᵢ, where d_{cyl}<<Dᵢ, Dᵢ<Dₒ, and d_{cyl}<<(Dₒ−Dᵢ), may provide a capacitance between the two rings represented by the following equation:

$${C}_{rings} ≅ \frac{{π}^{2} ( {D}_{o} + {D}_{i} ) {ε}_{0} ε}{2 {cosh}^{− 1} ( \frac{{D}_{o} − {D}_{i}}{2 {d}_{cyl}} )}$$

When the voltage difference between the ring-shaped electrodes is V_{rings}, the charge per length on either ring-shaped electrode may be represented by the following equation:

$${λ}_{cyl} = \pm \frac{2 {C}_{rings} {V}_{r i n g s}}{( {D}_{o} + {D}_{i} ) π}$$

Combining the three foregoing equations results in the following equation representing the electric field near either of the ring-shaped electrodes, in terms of the voltage and geometry:

$${\overline{E}}_{cyl} ( r ) = \frac{{V}_{r i n g s}}{2 r {cosh}^{- 1} ( \frac{{D}_{o} - {D}_{i}}{2 {d}_{cyl}} )} \hat{r}$$

Thus, for example, when d_{cyl}=0.6 mm, Dᵢ=50 mm, and Dₒ=100 mm, then the maximum electric field at the surface of either ring-shaped electrode is Ē_{cyl} (r=d_{cyl}/2)=4 V_{rings} volts/cm. This electric field drops off by 2× at a distance of 0.3 mm away from the electrode surface. Thus, a 6.8 KV voltage pulse would produce an electric field of 40 KV/cm to 50 KV/cm at a distance of 10 microns from the electrode.

The electric field strength from a line charge or cylindrical conductor may decay based on an inverse of the radial distance. To provide a more confined field, a linear array of point charges or spherical conductors may provide an electric field that decays based on the inverse square of the radial distance, for distances that are small compared to the spacing of the array, as set forth in the following equations:

$$\vert {E}_{c o m b} \vert = {\sum }_{n = - N / 2}^{n = N / 2} \frac{{q}_{sphere}}{4 π {ε}_{0} ε ( {z}^{2} + {( x - n {s}_{s} )}^{2} )}$$

$${\vert {E}_{c o m b} \vert}_{x = 0} = \frac{{q}_{sphere}}{4 π {ε}_{0} ε} ( {\sum }_{n = 1}^{n = N / 2} \frac{1}{{z}^{2} + {( n {s}_{s} )}^{2}} + \frac{1}{2 {z}^{2}} )$$

$${\vert {E}_{c o m b} \vert}_{x = s / 2} ≅ \frac{{q}_{sphere}}{4 π {ε}_{0} ε} {\sum }_{n = 1}^{n = N / 2} \frac{2}{{z}^{2} + {{s}_{s}^{2} ( n - 0.5 )}^{2}}$$

The sphere spacing ss and radii rs may give a field strength variation between the two extremes of x=0 and x=sₛ/2 at a depth of zₜᵣₑₐₜ=z−rₛ.

$${\sum }_{n = 1}^{n = N / 2} \frac{1}{{( \frac{{z}_{t r e a t}}{{r}_{s}} + 1 )}^{2} + {( n \frac{{s}_{s}}{{r}_{s}} )}^{2}} + \frac{1}{2 {( \frac{{z}_{t r e a t}}{{r}_{s}} + 1 )}^{2}} = 2 {\sum }_{n = 1}^{n = N / 2} \frac{2}{{( \frac{{z}_{t r e a t}}{{r}_{s}} + 1 )}^{2} + {( \frac{{s}_{s}}{{r}_{s}} )}^{2} {( n - 0.5 )}^{2}}$$

The above relationship holds for sₛ˜5 rₛ when zₜᵣₑₐₜ<0.1 rₛ.

Alternatively, an electrode array may be formed as a ring of hemispherical conductors about a common center, connected so that nearest neighbor electrodes have the opposite potential. Mid-way between two spheres, only the field in the azimuthal direction (tangent to a circle through the conductors) remains, as represented by the following equation:

$${E}_{c o m b} {\vert}_{\hat{θ}} = \frac{{q}_{sphere} {θ}_{{0}^{r} 0}}{4 π {ε}_{0} {ε ( {z}^{2} + {( {θ}_{{0}^{r} 0} / 2 )}^{2} )}^{3 / 2}}$$

θ₀ is the central angle between two adjacent spheres, and r₀ is the distance from the central axis to the center of each sphere.

The electric field strength may not be uniform throughout the sample volume undergoing IRE treatment. For example, the electric field strength may locally peak in regions of high curvature of the electrodes, or where the electrical conductivity locally changes in the sample volume. Because the temperature increase is related to the square of the electric field strength, such a local maximum field strength may only be about 10% to about 30% higher than an average field strength. The electric field strength may decay with distance from the electrodes. Efficacy for the IRE treatment of cells of a designated size may be considered to stop when the electric field strength drops to about 50%. This treatment depth may be represented by dₚₒᵣₐₜₑ.

Further, IRE treatment of even large cells may cease at a predetermined distance dₘₐₓ from the electrodes. The electric field at that transition distance may be represented by the following equation:

$${E}_{trans} = 2 V / {d}_{l a r g e c e l l} .$$

Assume that the external electric field strength in a sample follows the power law:

$$\overline{E} ( r ) = {( \frac{a}{r + a} )}^{p} {E}_{max}$$

p=2 for hemispherical electrodes, p=1 for hemicylindrical electrodes, and p=0 for planar electrodes (away from their edges). For other electrode geometries, like ellipsoids, the local radial dependence may have 0<p<2. Assuming Eₘₐₓ=1V/d_{cell}, then

$$\frac{E}{{E}_{p o r a t e}} = \frac{1}{2} = {( \frac{a}{a + {d}_{p o r a t e}} )}^{p}$$

$${d}_{porate} = ( \sqrt[p]{2} - 1 ) a$$

Thus, for hemispherical electrodes, dₚₒᵣₐₜₑ=0.4 a. For hemicylindrical electrodes, dₚₒᵣₐₜₑ=a. Accordingly, the radius where treatment of larger cells ceases may be represented by the following equation:

$$\frac{{E}_{t r a n s}}{{E}_{max}} = {( \frac{a}{a + {d}_{max}} )}^{p}$$

$${d}_{max} = a ( \sqrt[p]{\frac{{d}_{l a r g e c e l l}}{{d}_{c e l l}}} - 1 )$$

Assume that the cells outside the treatment volume that should not be affected are 5 microns across, and the cells inside the treatment volume are 1 microns across. Then, the depth where treatment of the large cells stops for hemispherical electrodes is dₘₐₓ=1.2 a. The depth where treatment of the large cells stops for hemicylindrical electrodes is dₘₐₓ=4 a.

The electric fields near linear electrodes with smooth sharp edges falls off as a/r, with a representing the radius of the sharp edge, and r representing the distance from the edge. The fields near sharp points falls off as (a/r)², with a being the radius of the point and r being the distance from the point. The electric field may be uniform between two planar electrodes away from their edges.

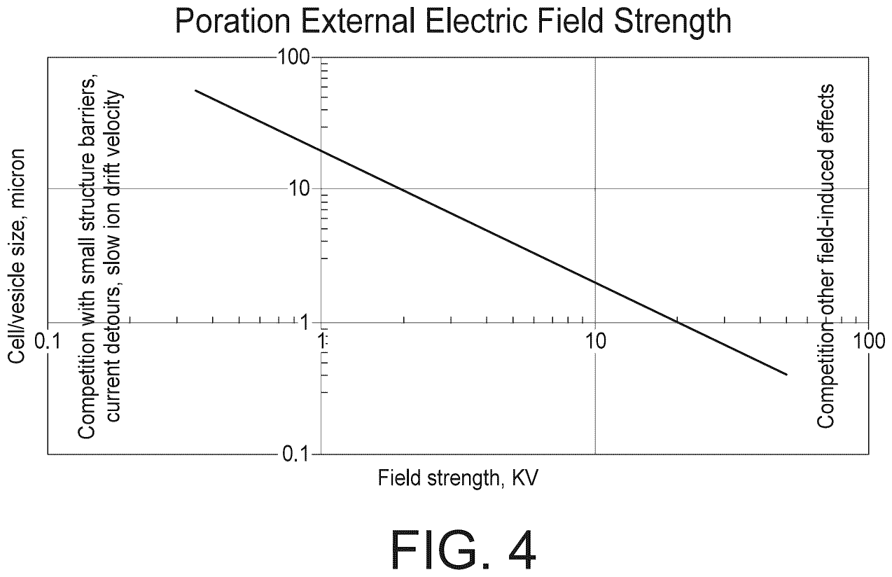

The graph of FIG. 4 shows electric field strength as a function of cell size, in accordance with aspects of the disclosure. FIG. 4 illustrates that an external electric field strength required to porate typical membranes is inversely proportional to the diameter of the membrane, as illustrated by the straight line on this log-log plot.

While it may be possible to porate large cells (above 50 microns) with low external fields (below 200 V/cm), this poration may be inhibited for the following reasons. The current path through most tissue may include many insulating membranes. For these cells to porate completely in one pulse, the number of these membranes multiplied by the voltage increase per membrane required for poration must be less than the externally applied voltage. If there is a distribution of spacings between the insulating membranes in series, the first membranes to porate will be those with the largest spacing. Multiple IRE pulses may be useful because the pulses may cause the sequential poration of wider-spaced membranes, which then exposes some membranes to wider spacings. 50 KV/cm electric fields may produce a potential difference of 1 V across membranes that are separated by a distance as small as 0.2 microns. The membrane charge up time may be approximately 0.5 ns, which may be faster than the charging time of the external electrodes (due to capacitive and inductive effects).

Tissue near the surface of the skin has an electrical conductance σₛ which varies from about 0.1/ohm-m to about 1/ohm-m. Taking 0.7/ohm-m as representative, applying 50 KV/cm to the tissue provides 17 Mwatt/cc to the tissue, which results in a rate of change in temperature of about 4 million deg C./s. However, limiting the total field application time to 25 ns limits the near-surface heating to a temperature rise of only 0.1 deg C. In general, if the tissue has a heat capacity Cᵥ (J/cc deg C.), the local heating rate may be represented by the following equation:

$${\dot{T}}_{cyl} ( r ) = \frac{{V}_{r i n g s}^{2} {σ}_{s}}{4 {r}^{2} {{C}_{v} [ {cosh}^{- 1} ( \frac{{D}_{o} - {D}_{i}}{2 {d}_{cyl}} ) ]}^{2}}$$

The maximum pulse duration to reach a target tissue heating ΔTₘₐₓ IS τₚᵤₗₛₑ=ΔTₘₐₓ/\dot{T}_{cyl}(d_{cyl}/2). The duration for bipolar pulses is τₚᵤₗₛₑ/2 for each monopolar component.

Because the local heating increases as the square of the field strength, a 10× rise in field strength would typically produce an unacceptable tissue heating even during a 25 ns pulse duration. Further, sharp edges on the electrodes should be avoided as the edges may cause overheating. Even the current crowding that occurs where a non-conductive mounting material partially masks an electrode ring may produce too much local electric field strength, and thus may cause overheating of the tissue.

Finite element analysis may be used to show that a semiconductive media adjacent to the electrode ring may even out this current crowding, creating a relatively uniform high field region in the half-space adjacent to the electrode. This is analogous, for example, to semiconducting films that are applied to conductor surfaces in high voltage power lines, which may reduce corona heating and power loss from imperfect conductor surfaces.

In this disclosure, the terms semiconductor, electrolyte, conductive media, and composite conductor in a polymer may be used interchangeably. The meaning of these terms may include a material with an electrical resistivity between 0.01/ohm-m and 100/ohm-m, which is greater than most metals, and less than most insulators. Either ionic or electric conductivity may be applicable, because voltage differences may be much larger than the ionization potentials at electrolyte-electrode interfaces.

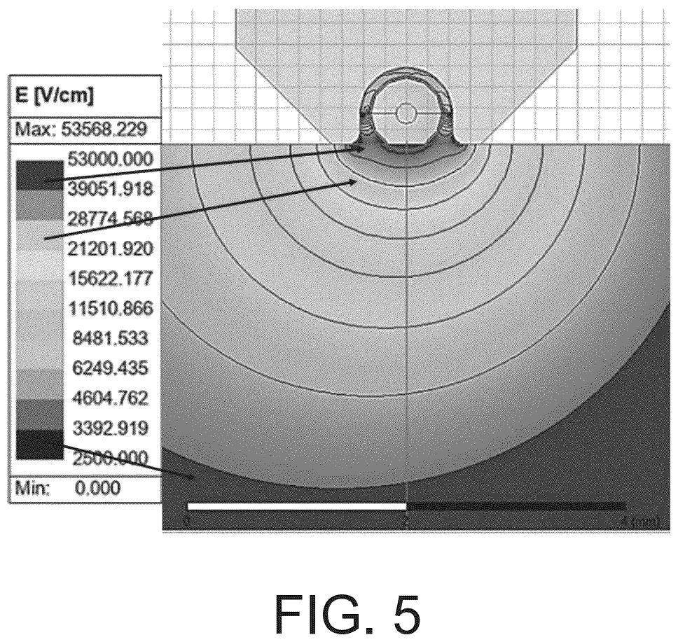

FIG. 5 is a depiction of an output of a finite element analysis model of an electrode applying an electric field to skin and tissue of a patient during IRE treatment, illustrating data from FIG. 4, in accordance with aspects of the disclosure. FIG. 5 shows that the cylindrical electrode may provide an electric field adjacent (e.g., at the location of) the electrode, which is over 50,000 V/cm, while the electric field may be less than 2500 V/cm at a distance of less than 2 mm away from the electrode (e.g., a short distance away from the electrode). The model is for an arrangement including the above-discussed two electrodes that are linear electrodes of 0.7 mm in diameter, with an adhesive that adheres the electrode to the base which is in contact with approximately a top half of the electrode and has a conductivity equal to that of tissue, with a spacing between the electrodes of 50.8 mm, with an excitation voltage applied to the electrodes of 15 KV, and with tissue material of the patient in contact with approximately the bottom half of the electrode. Electrolytic gel matched to the conductivity of the tissue fills gaps among the electrode, tissue, mount, and adhesive.

The model shows that the gel spreads out the field gradients so that there are no hot spots in the tissue at the junction of the electrode and the mount or adhesive. As FIG. 5 shows, |E| at a depth x=0 mm (tissue contact) is 41.9 KV/cm, |E| at a depth of x=1.18 mm into the tissue is 8.4 KV/cm, and a maximum |E| is 53.5 KV/cm.

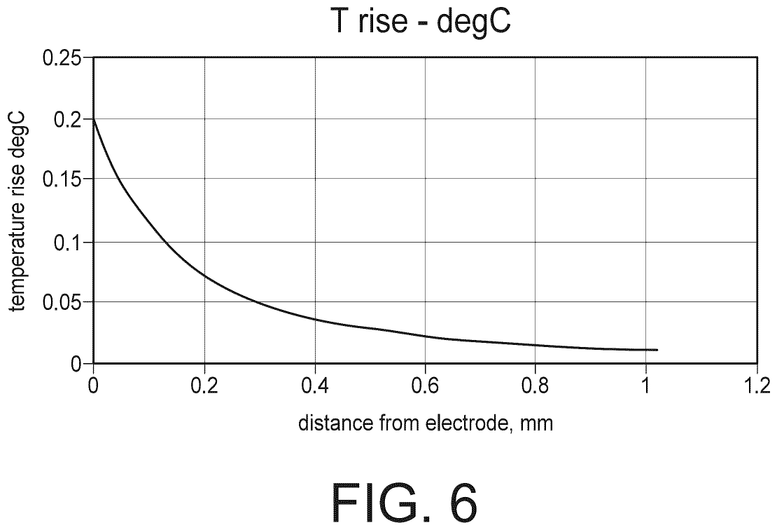

FIG. 6 is a graph depicting a relationship between temperature rise of the tissue undergoing IRE treatment, and a distance from the electrode, in accordance with the above discussion. As FIG. 6 shows, the temperature rise quickly falls off as distance increases.

The current required per excitation pulse may be equal to the electric field intensity times the conductivity times the treated area (per electrode). Assuming that the stratum corneum of the skin surface is arced through or abraded off, and that the skin conductivity is about 1 (ohm m), then a peak current into the 0.6 mm diameter 50 mm long electrode pair is 75 amps.

The power density dissipated in the tissue during the pulse may be equal to the current density square over the conductivity, which in accordance with the above example is 25 MWatt/cc. To control the temperature rise of the tissue, the pulse duration multiplied by this power density may be less than 5 joules/cc, and thus the pulse time should be less than 200 ns. Applying the peak power of 75 A*8.7 KV=652 KW at 600 Hz for 200 ns, results in a determination that the pulses require an average power of 80 milliwatts, which is a small power requirement. As a result of requiring relatively little power, the system is reduced in cost and complexity.

In accordance with the above example, the IRE treatment zone is about 1 mm wide for a 0.6 mm electrode. 10 Hz pulses allow the electrodes to be scanned at 1 cm/s while treating a 2.5 cm swath of tissue, in this example.

The thermal diffusivity of tissue may vary from about 0.1 to 0.16 mm²/s. Using the lower value, the time between voltage pulses may be 0.1 s for the heat to diffuse 0.1 mm in one direction between pulses, into the tissue. Scanning in one direction provides that tissue passing tangentially under the electrodes is exposes to the electric field several times in sequence. In some examples, the pulse rate may be increased if higher temperature rises may be tolerated.

Parallel electrodes with an impedance matching layer surrounding the electrode (such as saline or conductive adhesive or gel) may apply an electric field of uniform strength immediately adjacent the electrodes. With respect to a single-pulse high field strength limit, the combination may permit rapidly wiping areas of tissue and porating sub-micron structures near the tissue surface. In the calculation above, only the field strength associated with one of the two electrodes is presented, however when the system includes two electrodes, the field strength for the other electrode may be similar. With reference to FIG. 5 discussed above, when the system includes two electrodes, the finite element analysis for the other electrode may be a mirror image of that presented in FIG. 5. Treatment in accordance with the foregoing may be sufficient to result in small-organelle membrane destruction, which may kill surface bacteria colonies on the skin. These examples may be altered in both geometry and applied voltage, to target cellular structures at various depths, as would be needed for various clinical applications described herein.

The described IRE treatment results in true low thermal impact. A 30 ns (33.3 MHz) pulse at 50 KV/cm heats tissue about 0.1 deg C., for a treatment lethal to bacteria. Thus, the IRE treatment in accordance with the disclosure provides large area treatment at high electric fields with low heating, and without tissue burning. The IRE treatment also results in low nerve involvement due to high frequency bipolar pulses, which may prevent or cancel undesired nerve stimulation.

Further, the IRE treatment may use conductive media like saline, gel, or a conductor-loaded polymer that may include carbon in the lower portions of the gap between the electrode and skin, which may keep most of the steep electric field gradient away from the skin, preventing hot spots and burning of the skin or tissue.

In accordance with the above, a 50 KV/cm electric field may porate most all of the structures in a cell. In this disclosure, ‘pulse’ generally means ‘bipolar pulse’; the first half of a pulse's duration charges the electrodes +/−, and the second half −/+. Thus, a 50 ns pulse includes a 25 ns single-sided pulse followed by another pulse of the opposite polarity. The repetition rate fₚ of this biphasic pulse is set by the planned repeat pulse count Nₚ for a given area, the width of the uniform high-field region under the electrode Wₑ, and the scanning velocity vs of the electrodes over the skin, and may be represented by the following equation, fₚ=Nₚ Wₑ vₛ. fₚ, and which may be typically 10 Hz to 500 Hz.

The time for heat to diffuse out of treated skin or tissue may be on the order of a second, such as for shallow treatments. The rate that the electrodes may be moved to engage new regions of skin may be 10 Hz to 500 Hz. Therefore, temperature control may involve determining the pulse length and repeat pulse count so that the deposited energy density does not increase the temperature of the tissue more than about 2 deg C., in some examples.

As discussed, a cylindrical electrode pressed in contact with the skin may reasonably contact a swath width equal to the electrode radius. Thus, an electrode of length Lₑ would treat an area of Lₑ rₑ, and then may be displaced perpendicular to the electrode axis by about a distance rₑ to treat a next area on the skin. Accordingly, a 50 mm long electrode with a diameter of 0.6 mm may treat an area of about 0.15 cm² between positionings. Therefore, treating a square foot of skin may require about 6,100 positionings of the electrode on the skin. Thus, in accordance with some aspects of the disclosure, the electrodes may be electrically excited while being moved (e.g., slid or rolled) over the skin, to avoid the need to repeatedly position the electrode, perform an IRE treatment, reposition the device, and perform another IRE treatment, etc. A 600 Hz excitation for each position allows for a treatment rate of about 10 seconds per square foot of skin being treated.

Faster scanning with small tissue temperature rise is possible with one-dimensional scanning of linear electrode cylinder pairs spaced Sₚ apart. In this case, the fields may be represented by the following equation:

$${\overline{E}}_{p a i r} ( r ) = \frac{{V}_{p a i r}}{2 r {cosh}^{- 1} ( \frac{{S}_{p}}{{d}_{cyl}} )} \hat{r}$$

The heating rate may be represented by the following equation:

$${\dot{T}}_{p a i r} ( r ) = \frac{{V}_{p a i r}^{2} {σ}_{s}}{4 {r}^{2} {{C}_{v} [ {cosh}^{- 1} ( \frac{{S}_{p}}{{d}_{c y l}} ) ]}^{2}}$$

The treatment length in the scanning direction is approximately 2 d_{cyl}. When the pulse durations are τₚᵤₗₛₑ=ΔTₘₐₓ/\dot{T}ₚₐᵢᵣ (d_{cyl}/2), a scanning velocity v_{pair-scan} may be represented by the following equation:

$${f}_{s c a n} = \frac{{v}_{p a i r - s c a n}}{2 {d}_{cyl}}$$

For example, 25 cm/s scanning at 100% coverage may require 208 Hz pulses (or pulse pairs, for bipolar pulsing) with a cylindrical electrode having a diameter of 0.6 mm. Higher frequencies may allow for multiple treatments of the surface, with corresponding higher temperature rises. Multiple treatments without higher temperature rise may be accomplished with multiple scanning passes. A cylindrical electrode with a length of 2.5 cm may produce an aerial scan rate of 62.5 cm²/s.

As further discussed below, the rate at which voltage pulses are applied to the electrodes may be regulated by the observed velocity of the electrodes across the tissue surface. In some examples, the velocity may be measured with a measurement device (e.g., a roller) pressed against the tissue surface, which is coupled to an encoder, tachometer, or other structure that sends a velocity-dependent signal to the controller of the pulse generator. Other embodiments that involve surface velocity measurements may include optical imagers or speckle motion measurements, such as those which are used for two-dimensional computer mouse motion sensors. A possible implementation of the optical velocity measurements may use an image relay lens between the tissue surface and the sensor to provide electrical isolation from the high voltage discharge.

A handheld system in accordance with the disclosure may be convenient and provided at low cost, and may facilitate treatment in a clinical setting. Thus, for example, in an embodiment, an applicator may be in the form of a wand-like hand held device that a user would brush or otherwise move across an exposed tissue surface. The wand may detect a velocity and direction of tissue motion with respect to the wand, and may pulse the electrodes at a rate consistent with uniform surface treatment. For some indications the temperature rise in the tissue in combination with the desired electric field treatment may be elected, and in these cases the algorithms would be altered to increase the temperature to the desired degree, to facilitate the desired outcome. For example conditions that require collagen production are triggered with higher temperature cellular responses. Or if the cells that are being treated have a lipid and/or cholesterol composition that is better treated when temperature is elevated to effectively treat. The wand or gel used in the wand may temporarily color the tissue or skin surface when treated, to indicate the area of tissue that has been treated as well as to provide feedback to the clinician as to what area has been treated and how much treatment the area has received. Additionally, the system may be coupled with a robotic arm, for example, which may map the treatment zone as well as optimize the delivery accordingly.

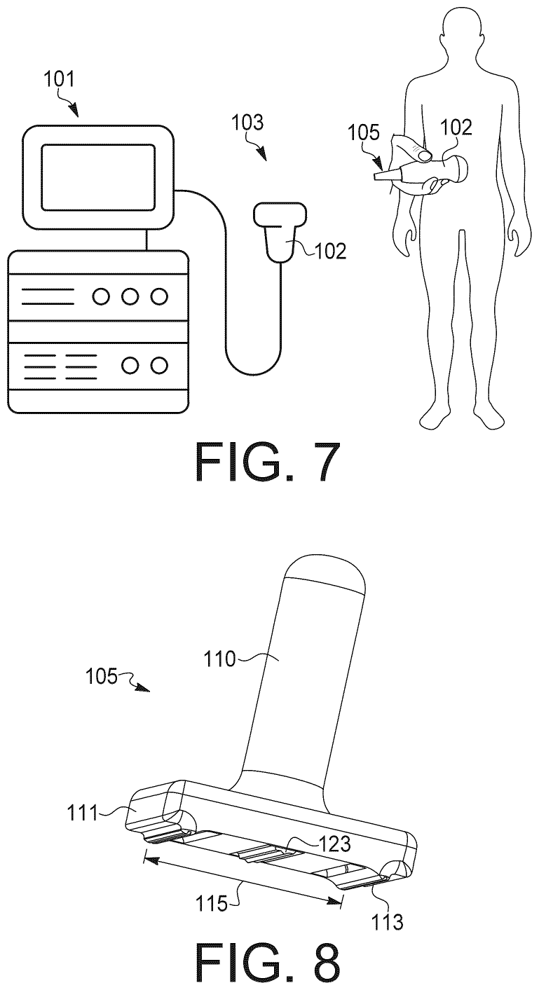

FIG. 7 depicts a schematic view of a treatment system 100 (referred to as the system 100) including a generator 101 and an applicator 102, which may be used to perform an IRE treatment, in accordance with aspects of the disclosure. The generator 101 may include a power source to provide power to the applicator 102, such that the applicator 102 may produce the electric field for IRE treatment of skin and tissue, as described. In some embodiments, the applicator 102 may be connected to the generator 101 by a cable. In other embodiments, the applicator 102 may be a self-contained applicator, which is not connected to a separate generator, such as the generator 101, and such that the applicator 102 is substantially all or all of the system 100. The applicator 102, or any of the other disclosed applicators, may be referred to as a hand-held applicator. The system 100 may allow for the selection of different algorithms, such as through operation or manipulation of the generator 101 or corresponding components of the applicator 102, to treat dermatology conditions. The applicator 102 may be suitable for treatment of multiple different dermatology conditions, or may be suitable for treatment of a specific condition. For example, the generator 101 or corresponding components of the applicator 102 may provide pulsed field ablation treatment in addition to IRE treatment, as described. In some examples, a voltage applied to the electrodes of the applicator 102, as further described below, may be from about 500 volts to over about 15,000 volts. The voltage may be provided by the generator 101 or batteries included in the applicator 102, such as in a handle of the applicator 102 for example. The power and communication between the wand and generator may include any known method in the art such as Bluetooth and other wireless methods.

FIG. 8 depicts an isometric view of an applicator or wand 105, in accordance with aspects of the disclosure. As FIG. 8 illustrates, the applicator 105 may include a handle 110 extending from a base 111, the base 111 retaining two electrodes 113 therein, which are separated from one another by a linear distance 115. In some examples, the base 111 or the handle 110 may be a nonconductive material, such as rubber, or another material, which suppresses surface arcs from forming between the electrodes 113. In some examples, as FIG. 8 illustrates, the electrodes 113 may be linear electrodes, and may be cylindrical in shape (e.g., having an about circular cross-section). In some examples, the electrodes 113 may be hemicylindrical, hemiellipsoid, semicylindrical, or semiellipsoid. In some examples, the electrodes 113 may be cylindrical electrodes having diameters of about 0.6 mm, and may be spaced about 2″ apart from each other. Either or both of a dimension or a shape of the electrodes 113, and/or the distance 115 between the electrodes 113, may be determined to provide different treatments with the applicator 105, as further described. As FIG. 8 also illustrates, the base 111 may include a motion detection device 123, discussed in further detail below. The applicator 105 and/or the generator, such as the generator 101, may also control and dispense conductive gel to help facilitate the procedure, as discussed below. A diameter of the electrodes 113 and the distance 115 may be designed specifically for different indications, as described herein.

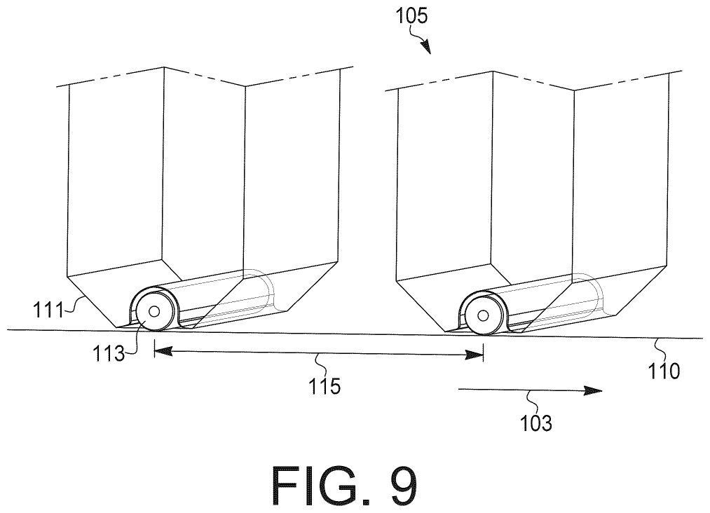

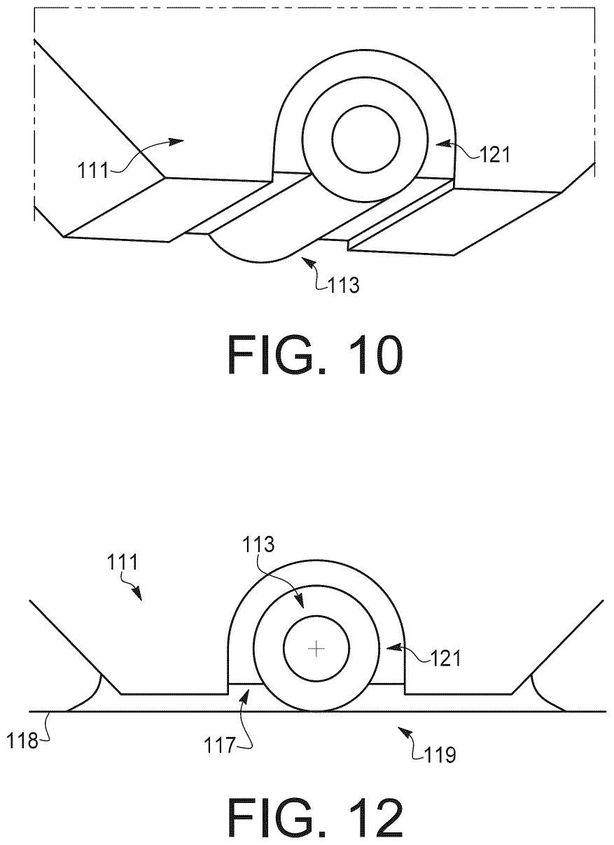

FIG. 9 depicts a detail view of portions of the applicator 105 against the skin 118 of a patient, the applicator 105 moving along a direction 103, while FIG. 10 is a detail view of one of the electrodes 113 retained within the base 111, in accordance with aspects of the disclosure. The direction 103 may be generally linear and about parallel to a surface of the skin 118, as well as about perpendicular to axes of the electrodes 113. As FIG. 10 shows, an adhesive 121 may retain the electrodes 113 within the base 111. In some examples, the adhesive 121 may be a nonconductive adhesive, as previously discussed, and the base 111 may be a formed from a nonconductive material like silicon or polyurethane, also as discussed. In some examples, such as when the adhesive 121 is insulative, there may be a gap between the base 111 and the electrode 113 which is not filled with the adhesive 121, thereby allowing gel on the surface of the skin 118 (discussed below) to fill the gap. In some examples, such as when the adhesive 121 is semiconductive, there may or may not be a gap between the base 111 and the electrode 113. This configuration may eliminate ion concentrations that could produce thermal hot spots.

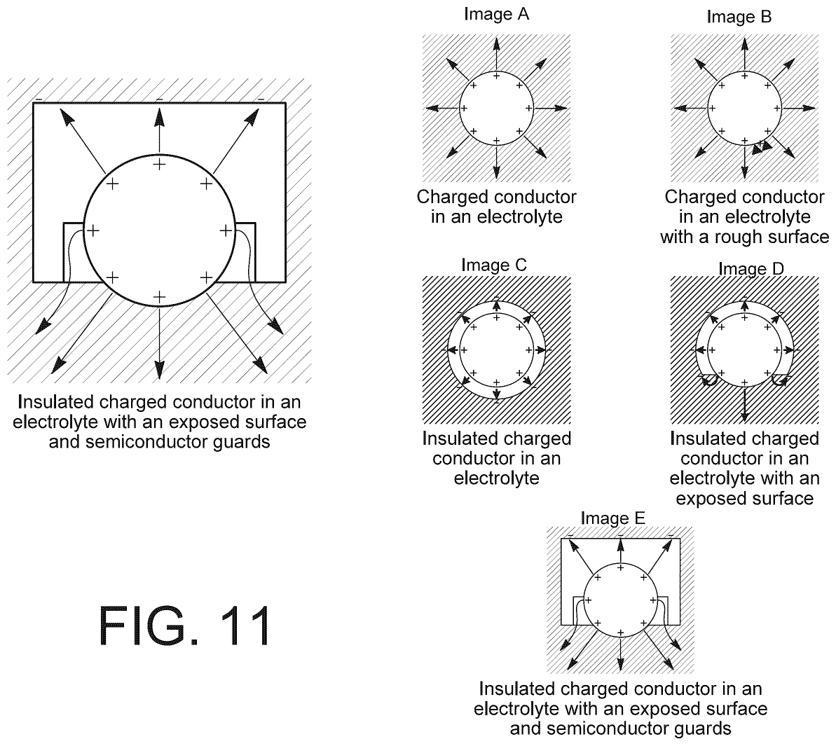

FIG. 11 shows images depicting different configurations of electric fields emerging from a spherical or cylindrical electrode. Image A, which illustrates a charged conductor in an electrolyte, shows a smooth metal surface of nearly constant curvature. The electric field lines are all radial, and their density is fixed by the radius. Image B, which illustrates a charged conductor in an electrolyte with a rough surface, shows a generally smooth metal surface for the electrode, except for a sharp asperity. The electric field lines are generally the same as in Image A, except for being much stronger and of higher density near the asperity; the density at the asperity fixed by the radius of the asperity. Image C, which illustrates an insulated charged conductor in an electrolyte, shows that the electrolyte acts as a conductor (which has no internal electric fields) if there is no current. Charge gathers on the surface of the surrounding insulating sphere, creating strong radial electric fields in the insulator, and none in the electrolyte. Image D, illustrating an insulated charged conductor in an electrolyte with an exposed surface, shows problems that may arise when an electrode in an electrolyte is partially surrounded by an insulator. The surface charge on the outside of the insulator creates a strong dipole with the surface charge on the electrode, creating a stronger electric field gradient at the joining of the insulator and the electrode than exists over the rest of the electrode. Image E, illustrating an insulated charged conductor in an electrolyte with an exposed surface and semiconductor guards, shows how the local electric field hot-spot of Image D may be dispersed by extending the electrolyte or semiconductor past the original joining of the insulator and the electrode of Image D. The grey semiconductor shown has approximately the same conductivity as the electrolyte or tissue. The lower current density through the semiconductor leads to a dispersed electric field gradient at the interface to the tissue.

This series of figures illustrates aspects of invention and shows that the hot spots adjacent to the cylindrical electrodes would go away when gaps adjacent to the electrodes are filled with gel. Image A is the ideal case of a smooth constant curvature electrode with no adjacent insulator, where no filler is needed. Image B is the practical case of a point (or roughness) on the electrode, the point causes the field to be locally intense. Image C is an insulator surrounding the electrode, where no filler needed. Image D is a partial insulator surrounding the electrode. The field is intensified at the junction of the insulation, the electrolyte, and the electrode. Image E is an implementation of the filler to resolve the high field condition of the middle right chart. Even for IRE electrodes for use on other than scanning skin, tissue damage can be avoided by adding semiconductor filler to the high field hot spots.

FIG. 12 depicts a further detail view of a portion of the applicator 105 against the skin 118 of the patient for treatment of bulk of tissue 119, according to some embodiments of the disclosure. As FIG. 12 illustrates, a gel 117 may be disposed on the skin 118, between the electrodes 113 and the skin 118. In some examples, the gel 117 may be a conductive gel that conducts energy from the electrodes 113 to the skin 118 more effectively than does air. In some examples, the gel 117 may disperse heat generated by the electrodes 113 more effectively than does air, and thus the gel 117 may reduce heating of the skin 118 during treatment with the system 100. The conductive media in the gel 117 is a poorer electrical conductor than most metals, but a better conductor than most insulators.

Image B of FIG. 11 is a representation of the theory behind the electrode, such as the electrode 113, and the semi-conductive element that facilitates the distribution of the electric field around the electrode in such a way that there are no hot spots. The concept of adding a resistive material to the high-field-gradient regions around the electrodes is similar to actions taken in the high voltage cable industry where a ‘semi-conductor’ layer is added around the HV core, mainly for corona suppression. The gel (or conductor-support composite material), such as the gel 117, acts like a semi-conductor. The gel or conductor is not a “semi-conductor” in the silicon sense that it may be doped, formed into junctions, and used as switches. However, the gel or conductor is a semi-conductor inasmuch as it is a poorer electrical conductor than most metals, and a better connector than most insulators.

The conductive media gel may also be a pharmaceutical, biologic, or drug delivery mechanism. The media gel may have any known medicinal chemical and/or biological agent infused thereinto. For example, the media gel may be mixed with an antibiotic agent, such as a penicillin family drug like erythromycin to facilitate a further desired treatment or effect. The media gel may include a burn cream such as aloe vera to facilitate healing. The media gel may have an acidic or basic element to help exfoliate the dermal skin layer and improve conductivity. Steroids also may be applied in this way.

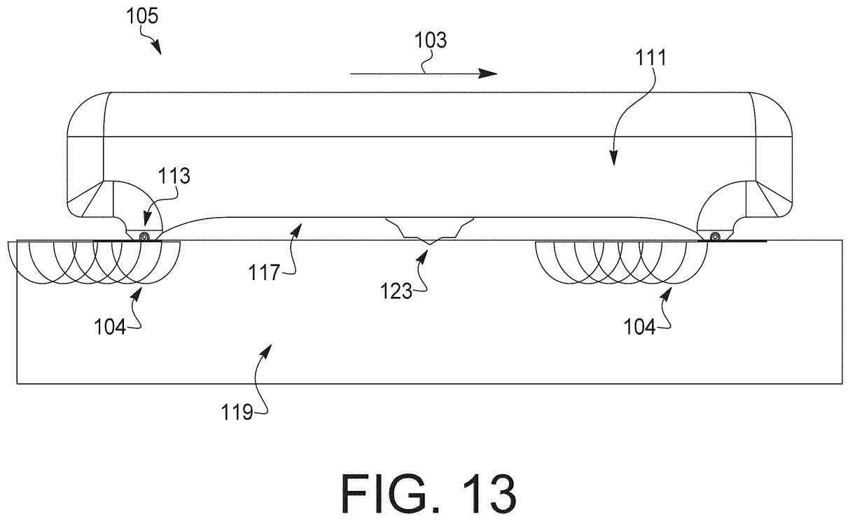

FIG. 13 depicts a side view of a portion of the applicator 105 including a positional translation device (e.g., the motion detection device 123), in accordance with aspects of the disclosure. The motion detection device 123 and may rotate on the skin 118 of the patient as the applicator 105 moves in the direction 103 across the skin 118. In some examples, the motion detection device 123 may include a spring biased wheel. Although FIG. 13 shows the motion detection device 123 as a wheel, the motion detection device 123 may be in the form of a scanning apparatus (e.g., a light scanning apparatus), a ball, or another type of tracking element, whether rotating or not.

As FIG. 13 shows, energy applied by the electrodes 113 may treat tissue regions 104 of the patient. The motion detection device 123 may provide locational or positional feedback, such as before, during, or after movement of the applicator 105 in the direction 103, for example. The applicator 105 may be programmed or otherwise controlled such that, regardless of the speed at which the applicator 105 is moved across the skin 118 of the patient, the applicator 105 may apply the appropriate treatment protocol to the tissue regions 104. This is because the applicator 105 may use the motion detection device 123 to determine the speed of movement of the applicator 105 across the skin 118, and the applicator 105 may deliver treatment of the tissue regions 104 based on the speed.

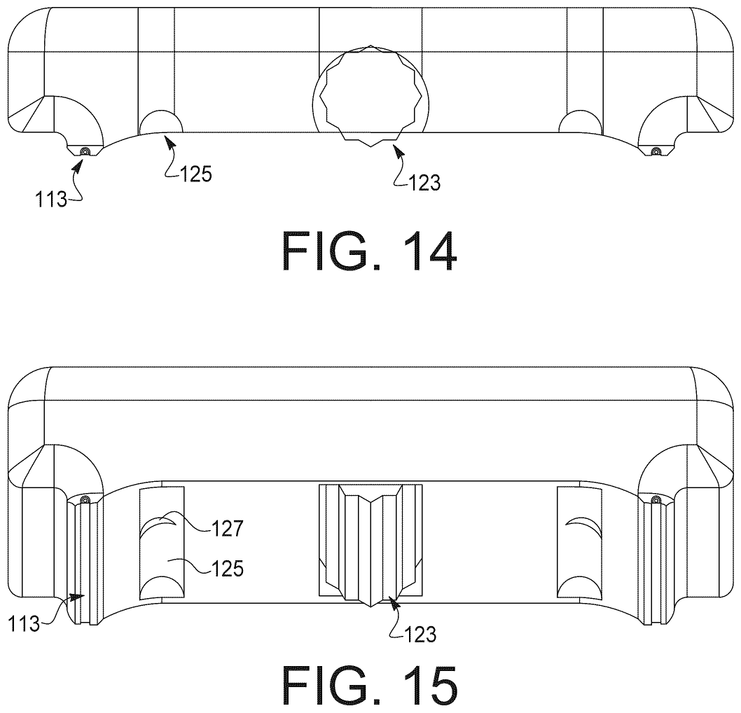

FIGS. 14 and 15 depict side and bottom isometric views of the applicator 105 including channels 125 having openings 127 that are in communication with the channels 125, in accordance with aspects of the disclosure. In some examples, the gel 117 may be delivered through flow paths that are in communication with the openings 127, such that the gel fills the channels 125. As discussed, the gel 117 then may be dispersed onto the skin 118 of the patient during treatment. In some embodiments, the applicator 105 may include one or more other flow paths and one or more additional channels, which may act as a vacuum to remove excess gel from the skin of the patient, or to keep the applicator 105 in contact with the skin 118 during treatment. The channels that deliver the gel and the remove the gel may be at various locations on the base 111—e.g., inboard or outboard of the electrodes 113, or other components of the applicator 105.

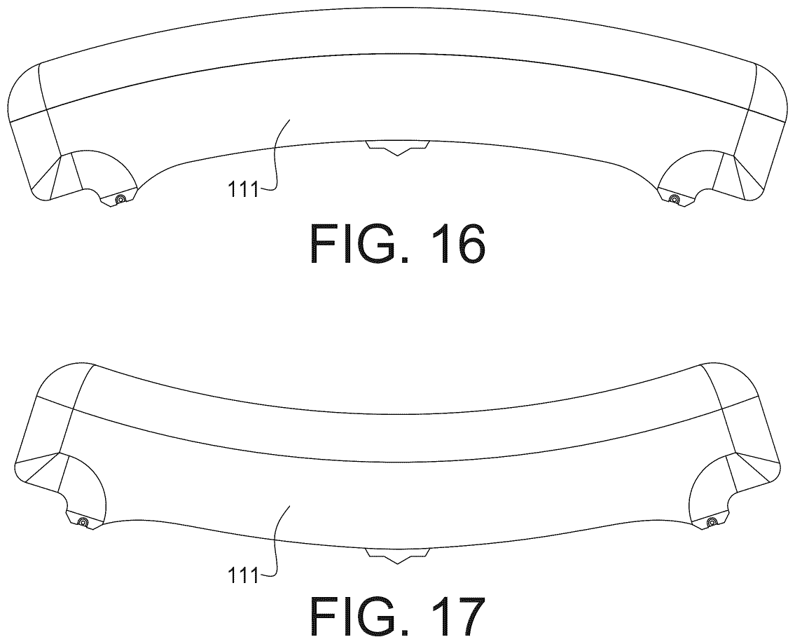

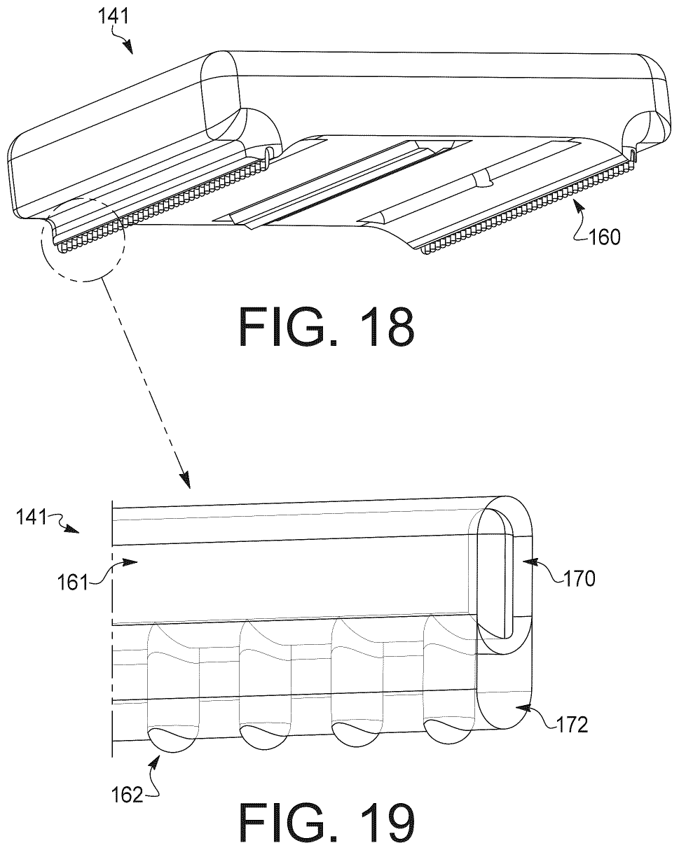

FIGS. 16 and 17 depict sides views of a portion of the applicator 105 in flexed configurations, in accordance with aspects of the disclosure. As FIGS. 16 and 17 show, one or more portions of the applicator 105, such as the base 111, may be flexible. For example, the base 111 may be flexed to concave or convex configurations, such that the contour of the base 111 may substantially match a contour of the skin 118 of the patient on which the base 111 is placed, during treatment with the applicator 105. In some examples, a material of the base 111 may include a flexible material, such as silicone polyurethane, or another polymer and elastomer combination. An extent to which the base 111 may flex in one or more directions may be based on an expected clinical application of the applicator 105, or the anatomy that the applicator 105 is designed to treat.

FIG. 18 depicts a bottom isometric view of an applicator 141, and FIG. 19 depicts a detail view of the applicator 141, in accordance with aspects of the disclosure. As FIGS. 18 and 19 show, the applicator 141 may include a plurality of electrodes 160 arranged in a comb or comb-like configuration. The electrodes 160 may extend from a shunting bar 161, which may be covered by an insulating material 170, the electrodes 160 may include ends 162 that are configured to contact the skin of the patient during the IRE treatment. As FIGS. 18 and 19 illustrate, the applicator 141 may include a resistive material 172, which may surround the insulating material (e.g., an adhesive) 170 as well as portions of the electrodes 160. In some examples, the electrodes 160 may be about cylindrical in shape, about 20 mil thick, and about 0.51 inches in length, while the ends 162 of the electrodes 160 may be about semi-hemispherical in shape. In some examples, portions of the electrodes 160, such as the ends 162 of the electrodes 160, may extend through the resistive material 172. In some examples, the applicator 141 may omit the resistive material 172, and in place of the resistive material 172 the applicator 141 may be used with gel. In some examples, the applicator 141 may include the resistive material 172 and may be used with a gel. In some examples, the gel and/or the resistive material 172 may fill the gaps between the electrodes 160, thereby reducing or eliminating hot spots that may other be caused by the electrodes 160 during the IRE treatment.

In some embodiments, the ends 162 of the electrodes 160 may be spaced such that a distance between adjacent ends 162 is about 0.5 times, about 1 time, about 2 times, about 2.5 times, or about 3 times a diameter of the ends 162. In some embodiments, a length of the electrodes 160 may correspond to a depth to which treatment is to be applied to the skin or tissue of the patient. In some examples, the resistive material 172 may have about a same conductivity as the skin of the patient. In accordance with some aspects of the disclosure, the configuration of the electrodes 160 shown in FIGS. 18 and 19 may provide a treatment field that may decay at a shorter distance from the electrodes 160 as compared to other electrode arrangements (e.g., the arrangement of electrodes 130 in the applicator 105), which may allow for a higher voltage field to be applied to the skin of the patient at shallower depths, which may be used to treat bacterial infections for example.

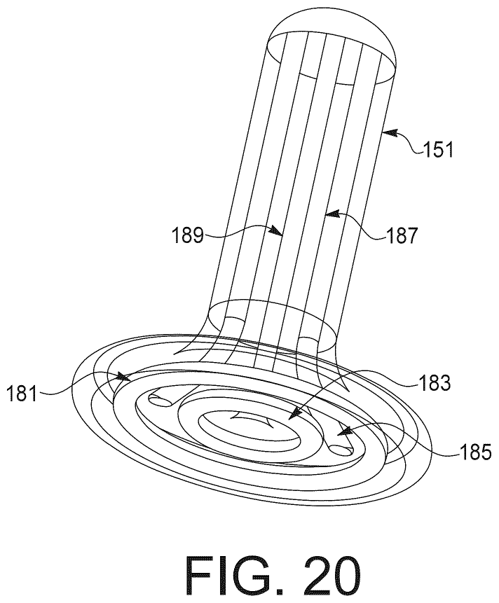

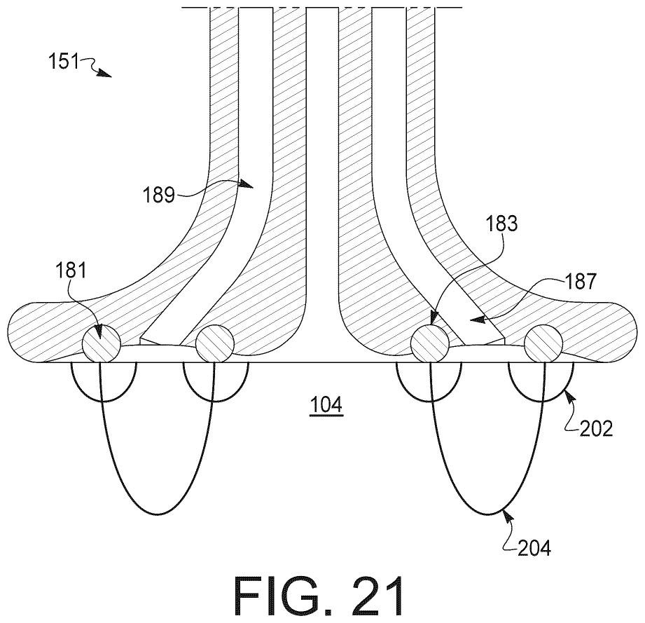

FIG. 20 depicts a bottom isometric view of an applicator 151, and FIG. 21 depicts a cross-sectional view of a portion of the applicator 151 applying an electric field to the skin of a patient, in accordance with aspects of the disclosure. As FIGS. 20 and 21 show, the applicator 151 may be a dual-ring application with an inner electrode 183 and an outer electrode 181. The inner electrode 183 and the outer electrode 181 may be spaced apart a distance equal to about 1 time, about 1.5 times, about 2 times, about 2.5 times, about 3 times, about 3.5 times, about 4 times, or about 4.5 times a diameter of the inner electrode 183 or the outer electrode 181.

As FIGS. 20 and 21 show, the applicator 151 may include one or more flow paths 187 each terminating in an opening 185, through which a gel (e.g., the gel 117) may be delivered through the applicator 151 to the skin of the patient.

As FIG. 21 shows, the inner electrode 183 and the outer electrode 181 may be sized and positioned to deliver the appropriate voltage field to treat the desired tissue type, resulting in both i) a near field effect into the tissue region 104, shown as a treatment field 202 as well as ii) a deep field effect, shown as a treatment line 204. Along the treatment line 204, the treatment is not uniform. The density of the field (V/cm) may change everywhere along the treatment line 204, such that it is not a line of constant treatment. In some examples, an electromagnetic deep field effect is oriented in a vertical or horizontal direction, as well as a tangential direction throughout the circumferential orientation, thereby subjecting cells to an electromagnetic field from multiple directions, including the X direction, the Y direction, and the Z direction. The applicator 151 may treat tissue at depths from about ½ millimeter to about 15 mm, and without causing thermal damage. In some embodiments, the pulse rate may be controlled to be about 10 Hz to about 20 Hz, which may allow heat to diffuse down into the tissue without causing thermal damage, for example.

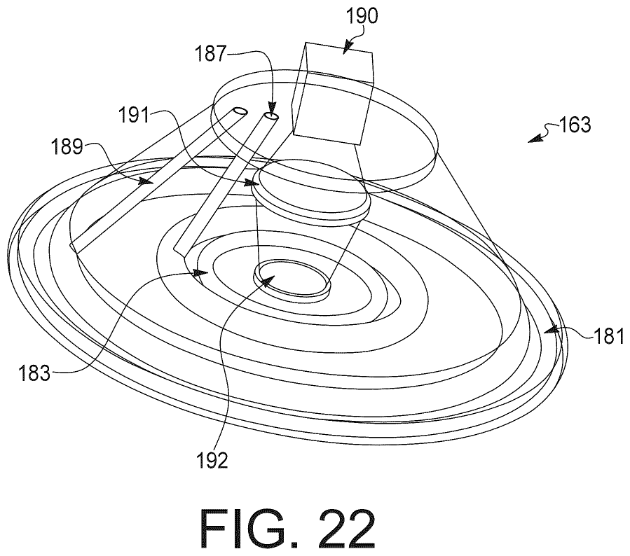

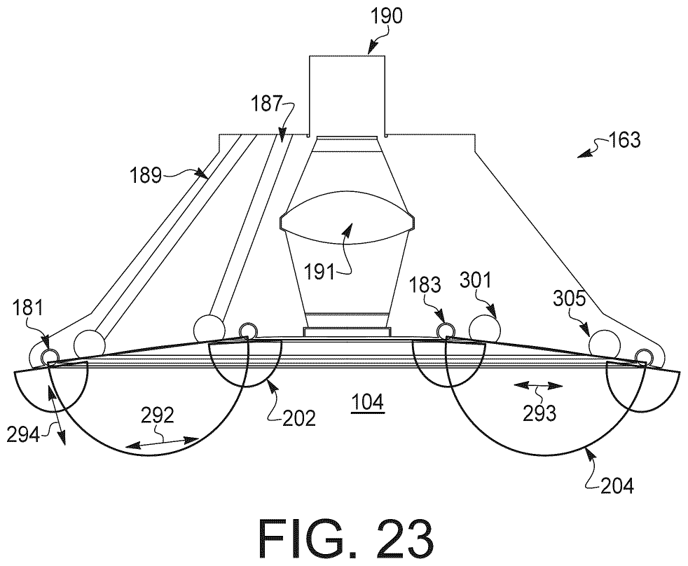

FIG. 22 is a top isometric view of a portion of an applicator 163, and FIG. 23 is a side cross-sectional view of the applicator 163 applying an electric field to the skin of a patient, in accordance with aspects of the disclosure. The applicator 163 may include optical sensor components, as further described, which may detect movement of the applicator 163, and which may control the application of electrical energy in response to the detected movement. For example, as FIGS. 22 and 23 show, the applicator may include optical sensor components including an optical window 192, a relay lens 191, and a splitter cube 190, which may provide for illumination and sensing, as described. In some examples, the optical window 192 may contact the skin of the patient through the conductive gel. The relay lens 191 may transmit illumination from a beam splitter to the optical window 192, and the resulting image of the skin may be formed by the relay lens 191 on a detector array at the splitter cube 190. Frame-to-frame shifts in image position may be interpreted as motion of the skin with respect to the inner electrode 183 and the outer electrode 181.

As FIG. 23 shows, the inner electrode 183 and the outer electrode 181 may be sized and positioned to deliver the appropriate voltage field to treat the desired tissue type, resulting in both i) a near field effect shown as the treatment field 202 into the tissue region 104, as well as ii) a deep field effect shown as the treatment line 204. In some examples, an electromagnetic deep field effect is oriented in a vertical or horizontal direction, as well as a tangential direction throughout the circumferential orientation, thereby subjecting cells to an electromagnetic field from multiple directions simultaneously. For example, the directions 292, 293, and 294 show locally favored orientation of cells for poration. The applicator 163 may treat tissue at depths from about ½ millimeter to about 15 mm, without causing thermal damage.

In accordance with some aspects of the disclosure, the electric field direction may vary over 4pi steradians depending on the radial orientation of the electric field. In some examples, if there are long cells (like muscle fiber) that respond strongly at one arbitrary orientation, over-scanning with the applicator 163, for example, may facilitate treatment.

As discussed, gel may be pumped through the flow paths 187. In some embodiments, a pump such as a peristaltic or piston pump may be connected to the applicator 163, to pump the gel through the flow paths 187. The flow paths 187 may be designed, including the openings at the ends thereof, such as facilitate movement of the applicator 163 over the skin, as well as to effectively deposit the gel on the skin. In some examples, the flow paths 187 are connected to channels formed in a bottom surface of the applicator 163.

In some embodiments, one or more of the optical sensor components may be affected by the gel. The optical sensor components may be configured to operate at a gel interface. In some example, one or more of the optical sensor components may be positioned on a perimeter of the applicator 163, and some of the optical sensor components may operate at a gel interface, while some of the optical sensor components may operate at an air interface. In some instances, one or more of the optical sensor components may be spaced apart from the gel interface.

As discussed, one or more components of the applicator 163 may be formed from an elastomeric material, which may conform to the skin of the patient.

The applicator 151 may include the flow paths 189 that may act as a vacuum, which may vacuum excess gel from the skin of the patient. The vacuum provided by the flow paths 189 may tend to hold the applicator 163 against the skin of the patient. In some examples, the flow paths 189 are connected to channels formed in the bottom surface of the applicator 163. When gel is pumped into the applicator via the flow paths 187, a peristaltic pump applying either a vacuum or positive pressure may be used. In one examples, an inner collar is linked to a gel reservoir, and an outer collar is linked to the peristatic pump, which may exit to a filter that only allows air to pass, as well as to a recirculation back to the reservoir.

A toroid or half-toroid groove or trench 301 outside of the inner electrode 183 may encourage the gel to flow tangentially with low resistance, so there will be less likelihood of dry spots. That half-toroid groove or trench 301 may only be fed by one or two of the flow paths 187.

The flow resistance experienced by the gel feed pump may correspond to the biggest gap being fed gel. Separately, the overall axial force applied to the applicator may give an average psi under the applicator. This pressure may be monitored and should be above a threshold before the high voltage is applied. One or more of the flow paths 187 and/or 189 may have a constriction at its exit that roughly matches the expected flow resistance over the skin as the applicator is scanned. This is a rule-of-thumb from air-bearings for providing a stable interface layer thickness. A second toroid or half-toroid groove or trench 305 disposed to the inside of the outer electrode 181 may act as a vacuum, removing the excess gel. This may allow considerable excess gel to be deposited during scanning. As discussed, the vacuum may both tend to hold the applicator against the skin, and give feedback as to whether portions of the applicator have lifted off the skin.

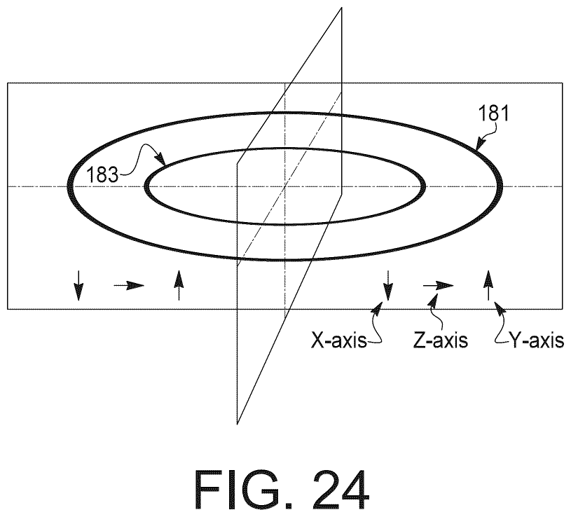

FIG. 24 depicts a schematic isometric view of the three-dimensional electric field produced by the inner electrode 183 and the outer electrode 181, in accordance with aspects of the disclosure. As FIG. 24 shows, at a full treatment depth, the electric field direction rotates depending on the radius from the center. This enables long-cell-dimensions to be engaged in arbitrary directions, including the X direction, the Y direction, and the Z direction. Between the electrodes, the deep field may not have a z component, while under each ring the electric field may be mostly in the z direction.

The plots of |E| have been ignoring the direction of the field. The cells respond to that direction. One advantages provided by a scanning electrode is that it may (accidentally) sweep the electric-field direction, not just parallel to the skin surface (e.g., combinations of X-axis and Y-axis directions) but also perpendicular (e.g., Z-axis direction). Between the electrodes 181 and 183, the deep field does not have a Z component, while under each electrode the electric field is mostly in the Z-axis direction.

FIG. 24 shows that the field direction at depth half-way between the rings follows a radial pattern, while the field direction under the rings is vertical, so that all cell orientations can be engaged without having to rotate the electrodes. All possible orientations of the electric field relative to cell shape and position may be achieved. At the full treatment depth, the field direction rotates depending on the radius from the center. This enables long-cell-dimensions can be engaged in arbitrary directions. Cells of different shapes will require different field concentrations to porate. For these configurations, when the electrodes are far apart compared to their diameter, the treatment depth is determined by their diameter. To maximize treatment depth, the spacing between the electrodes may be reduced to 2.5-3× their diameters with a larger diameter electrode.

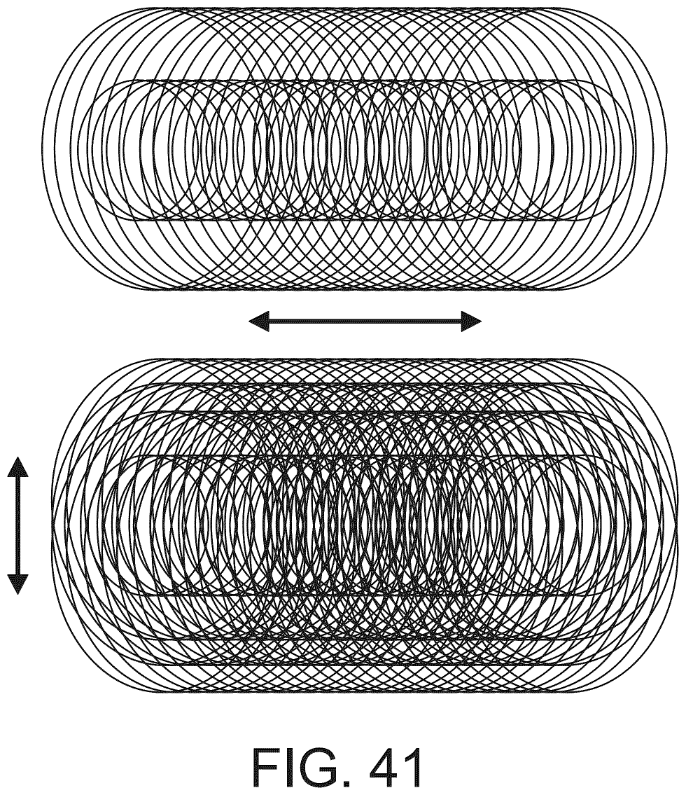

The electric-field direction varies over 4pi steradians depending on the radial orientation; if there are long cells (like muscle fiber) that respond strongly at one arbitrary orientation, overscanning with this geometry is more likely to treat them, such as shown in FIGS. 41-43.

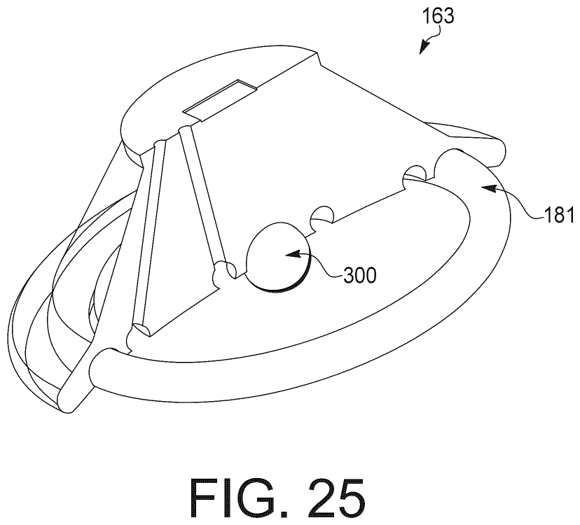

FIG. 25 depicts a partial cross-sectional view of another embodiments of the applicator 163, in accordance with aspects of the disclosure. As FIG. 25 shows, the applicator 163 may include an electrode 300 which is substantially spherical in shape, in place of the inner electrode 183. The electrode 300 may roll or slide over the skin of the patient during the IRE treatment with the applicator 151.

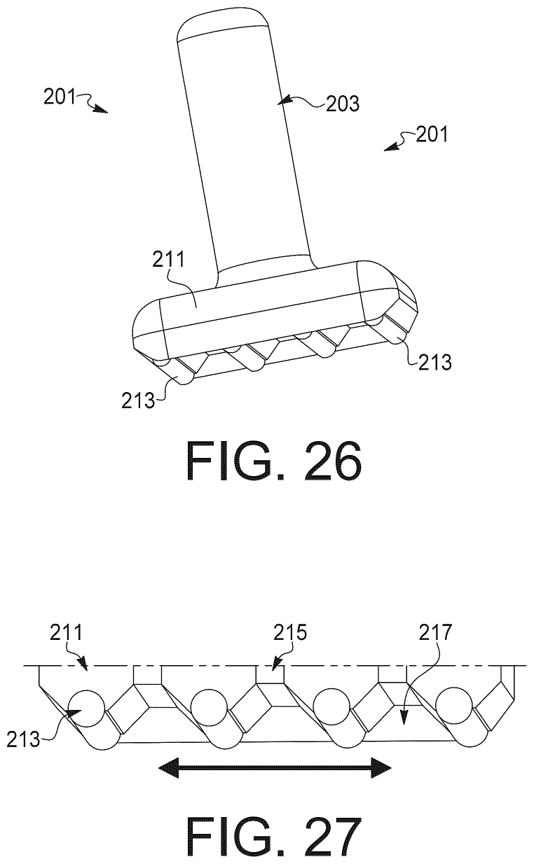

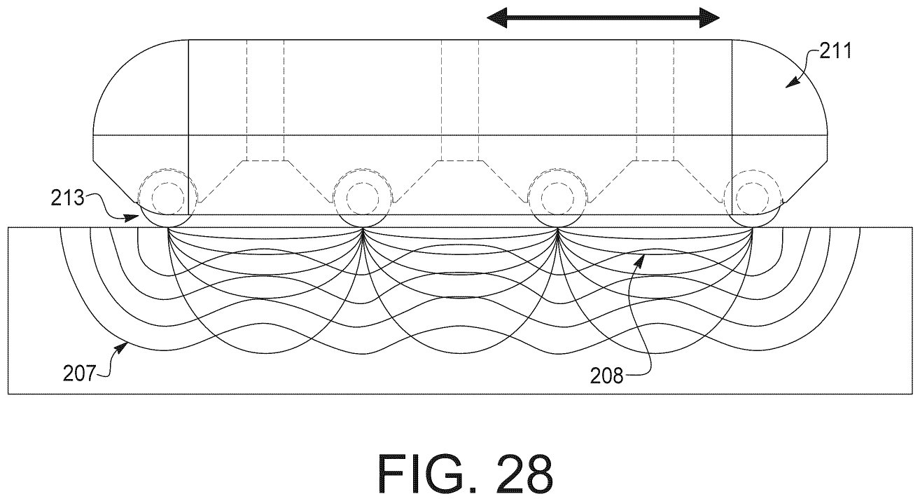

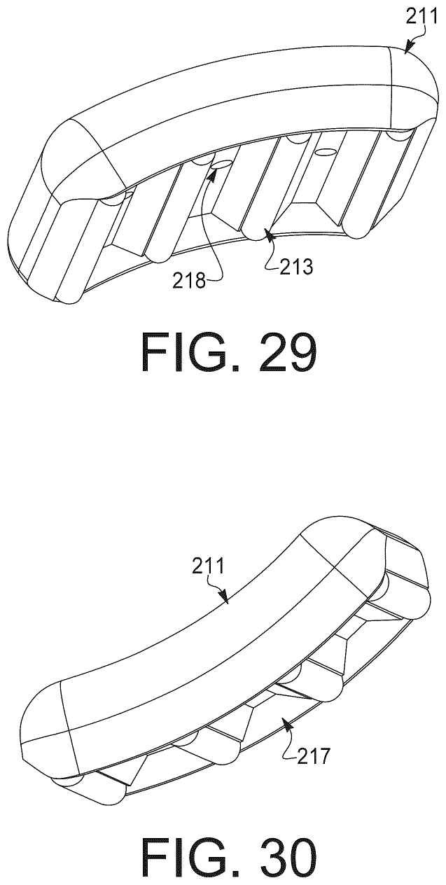

FIGS. 26-30 depict views of an applicator 201, in accordance with aspects of the disclosure. As FIGS. 26-30 show, the applicator may include a handle 203 and a base 211, which may include electrodes 213 which are larger than electrodes 113 of previous embodiments. The spacing between the electrodes 213 is about 2, 2.5, 3, 3.5, or 4 times the diameter of the electrodes 213. This arrangement may provide deeper treatment into tissue 119 and may traverse the surface of the tissue 119 between which the electric field passes, as indicated by the treatment field 207 and treatment field 208, for example. Although this embodiment shows four of the electrodes 213, the applicator may include a greater number or a lesser number of the electrodes 213. In some embodiments, the number of the electrodes 213 may provide treatment over a 2 inch square area of the skin of the patient. As FIGS. 29 and 30 illustrate, the base 211 may be formed from an elastic material, so that the base 211 may flex and conform to the anatomy of the patient during treatment, as discussed. Further, the applicator includes gel delivery troughs 215 and gel occupational voids 217. Thus, for example, gel may be delivered into the troughs 215 through flow paths terminating in opening 218, and a volume of gel may occupy the voids 217. One advantage is the provision of a deep field effect that is possible over the small diameter surface electrodes described earlier. Another advantage is the ability to treat 15 mm deep into tissue while maintaining a surface temperature increase of less than 4 deg C. without external cooling agents. This design could incorporate active cooling to further drive the treatment type and depth. The gel could be chilled allowing for longer treatment times and higher voltage applications.

FIG. 31 is a graph of electromagnetic field strength versus the distance from the electrode in volts per centimeter, as well as a graph of an associated temperature rise versus a distance from the electrode, in accordance with aspects of the disclosure. The information on the graph was determined based on evaluation of an applicator with two electrodes each having a diameter of 0.6 mm and a linear length of 25 mm, a spacing between electrodes of 7 mm, tissue having an electrical conductivity of 0.7/ohm-m, tissue having a heat capacity of 2 joule/cc deg C., applied voltage of 10,000 V, a pulse frequency of 200 Hz, a manual scanning speed of 50 mm/s and a tissue thermal diffusivity of 0.13 mm²/s. The graph shows that the electric field decays at 1/r from the center of the cylindrical electrode, and the temperature rise decays at 1/r².

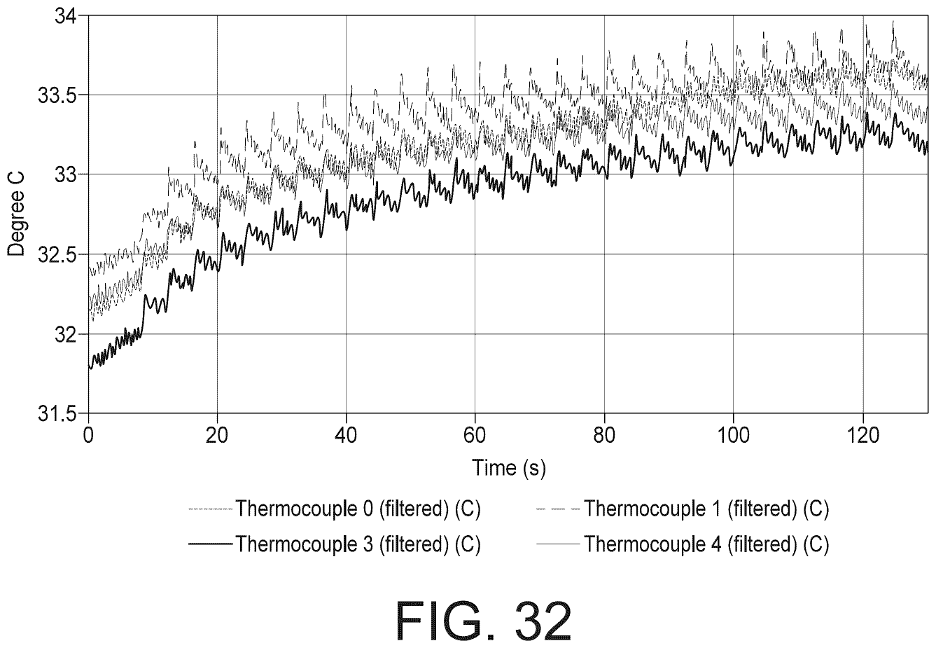

FIG. 32 depicts a graph of temperature versus contact time measured at an interface between the electrode and the skin, measured at four different locations, based on animal testing, in accordance with aspects of the disclosure. The testing includes treatment 30 times over 120 seconds, and thermocouples measure the resulting temperature rise. FIG. 32 shows that the rise in temperature is less than two degrees Celsius, although the individual treatments produce about 0.2 deg C. rise each. In some examples, treatments from multiple bursts of energy result in even less temperature rise. In some examples, the treatments using bursts of energy resulted in a temperature rise of about 0.4 degrees C. The algorithm provided for treatment using 5000 V, 30 pulses per burst, for 30 bursts. An unassisted cooling rate between bursts was about 0.1 deg C./second. In some embodiments, the cooling rate may be increased by adding a cooling feature into the applicator, which may allow more energy to be delivered into the tissue without a resulting temperature rise that may damage tissue.

It was further observed during animal testing that the applied energy did not adversely affect other bodily systems within the animal, such as the heart. Specifically, cardiac signals were monitored during therapy delivery and no transient or permanent changes in actual cardiac waveforms from therapy were observed. This graph demonstrates the only truly non-thermal application of IRE.

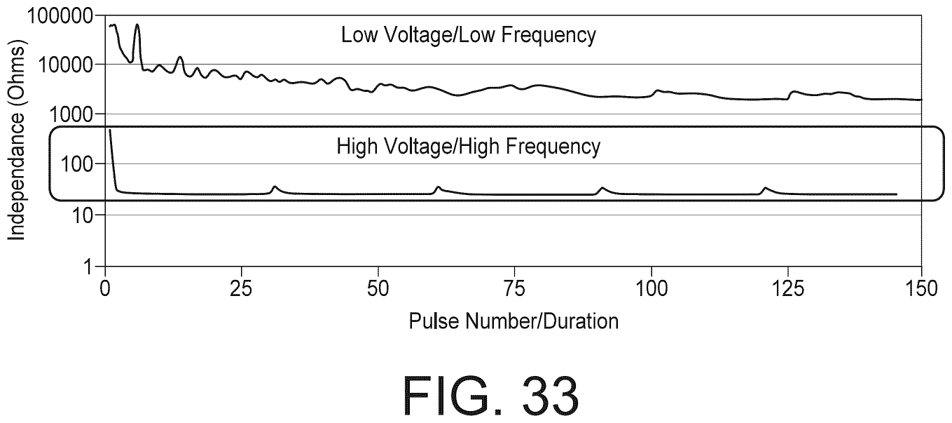

FIG. 33 is a graph showing impedance versus pulse number demonstrating the difference between a high and low voltage application in a graphic form, in accordance with aspects of the disclosure. FIG. 33 illustrates that a higher voltage applied results in a lower impedance. Impedance may be effected by the electrode (e.g., a surface area), a voltage, and a pulse. Both high frequency and high voltage mitigates the stratum corneum impedance of the dermal layer. Impedance is driven largely by the surface area of the electrode, the larger the volume of tissue we are driving energy into the lower the impedance. Tissue, including the stratum cornea, has a bulk resistance, and thus more tissue is treated as more resistors in parallel and thus lower impedance. Pulse repetitions may tend to form conduction channels through the stratum corneum, which then significantly reduce the effective impedance between electrodes applied to the skin. FIG. 33 indicates the immediate formation of conduction channels in the stratum corneum when fields on the order of 20 KV/cm are applied, as opposed to the about 100 pulses required to form conductive channels when lower fields on the order of about 1 KV/cm are applied. A large difference in the impedance of the tissue was demonstrated with more than a 3400 Ohm difference, as 3500 Ohms was the value for the lower voltage low frequency algorithm with an electrode designed for a 1250 V application, verses only 26-36 Ohms resistance for the high voltage, high frequency electrode and algorithm set for 15,000 V application.

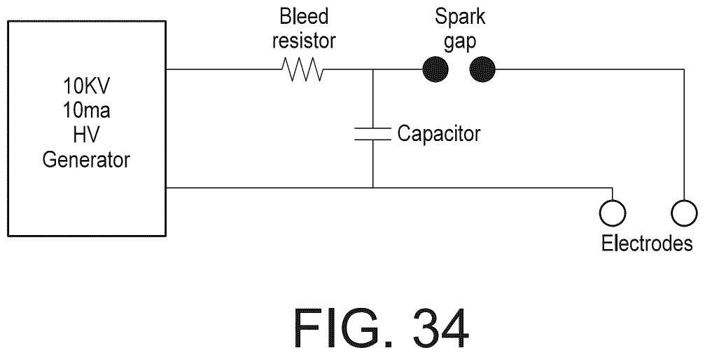

FIG. 34 is a schematic depicting a generator that may be used in IRE treatment, in accordance with aspects of the disclosure. As FIG. 34 shows, the generator may be a 10 KV, 10 ma, high voltage generator, and may include a bleed resistor, a spark gap, a capacitor, and electrodes for application of the electric field to the patient. The peak voltage of the generator may be determined by the geometry of the spark gap. The energy that is available is half the capacitance times the square of the peak voltage. This circuit may produce a bipolar oscillatory waveform with an exponentially decaying envelope. The net charge may be unipolar.

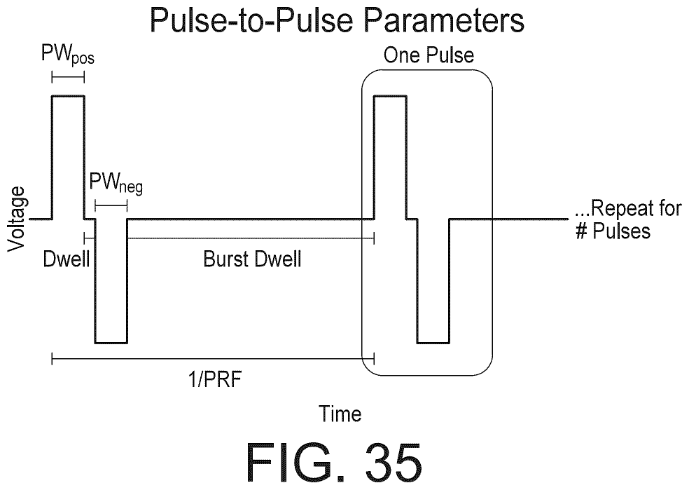

FIG. 35 shows two of a sequence of bipolar pulses having a relatively short dwell or off time between pulses in a bipolar pair, in accordance with aspects of the disclosure.

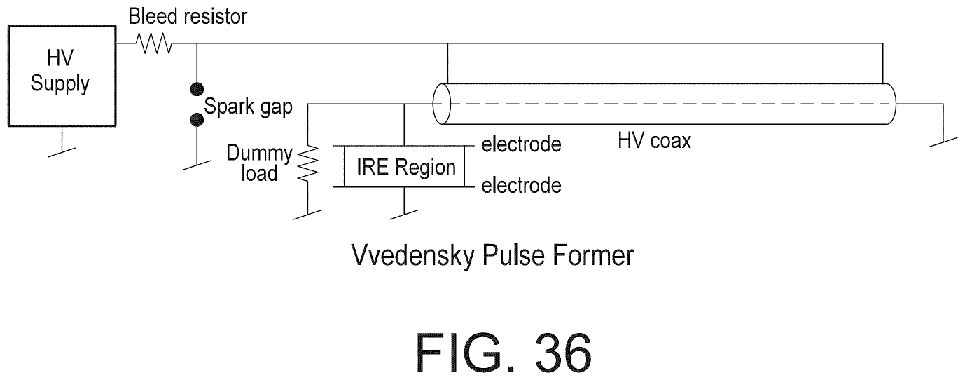

FIG. 36 shows a Vvedensky pulse former, in accordance with aspects of the disclosure. A Vvedensky pulse former is a bipolar pulse generator including a delay line pulse former, which turns a unipolar pulse into a bipolar pulse pair. Although the peak power for an applicator may be about 10 Kwatts to about 100 Kwatts, the average power delivered to the tissue may be a maximum of about 0.3 watts, and these power levels may be provided by a battery. Thus, in some examples, the pulse generator of FIG. 36 may be integrated into various ones of the applicator described herein, including the applicator 105. When the tissue sample is impedance matched to the coaxial delay line, a resulting waveform at the tissue looks like the graph of FIG. 35, where PWₚₒₛ=PW_{neg}=the delay time through the coaxial line, the dwell time is nearly zero, and the pulse pair period is about equal to the bleed resistance times the capacitance. Thus, modifying the high voltage supply output or the bleed resistance allows control of the pulse pair rate to match the scan rate.

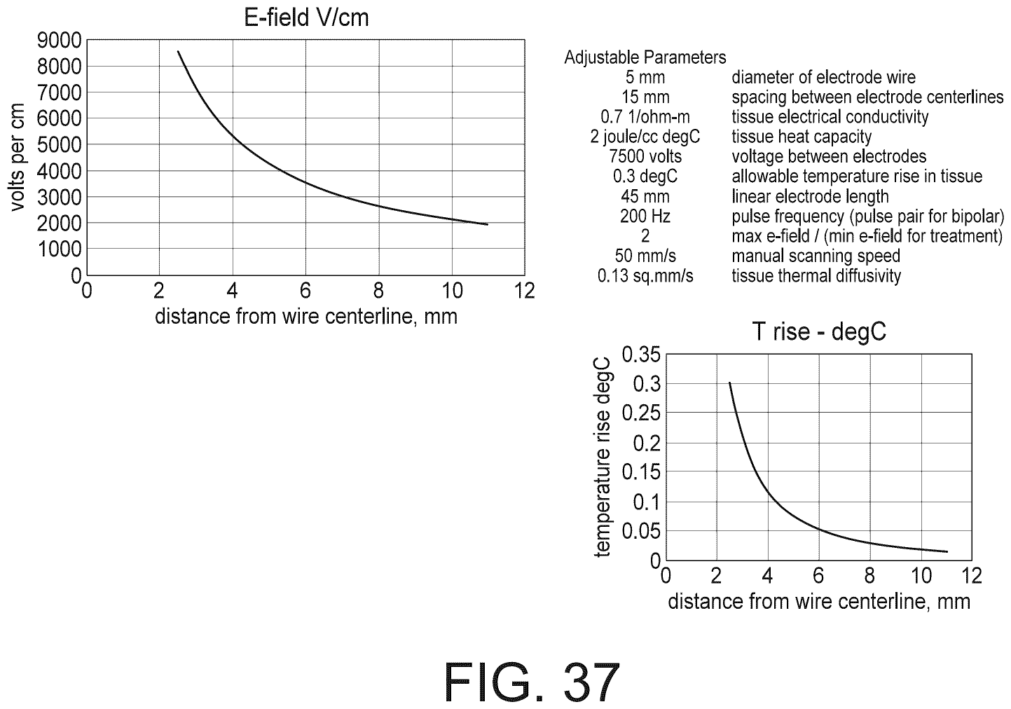

FIG. 37 is a graph depicting electromagnetic field strength versus the distance from the electrode in volts per centimeter, as well as a graph of an associated temperature rise versus a distance from the electrode, in accordance with aspects of the disclosure. The electrode configuration includes electrodes with a diameter of 5 mm, a spacing of 15 mm, driven by a drive voltage of 7500 volts. As FIG. 37 shows, the electric field at 12 mm is still approximately 2000 volts per centimeter, with a temperature rise of under 0.3 deg C. Thus, the skin or tissue is not thermally damaged by the configuration. This configuration is similar to one used to treat the animals whose data is depicted in FIGS. 31, 32, and 38-40.

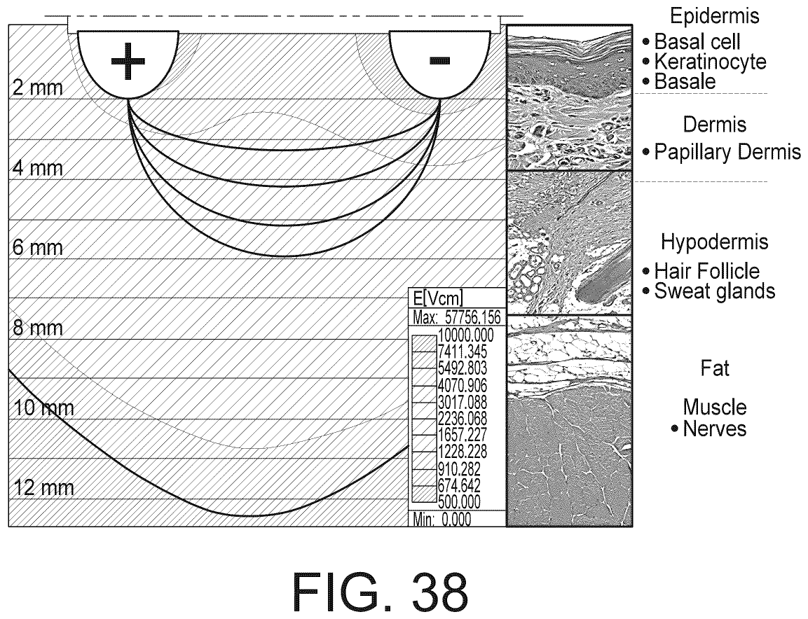

FIG. 38 illustrates a finite element analysis of the electrodes on tissue, indicating the depth of treatment, the image on the left side shows modeling of field intensity in tissue of design used, and the image on the right side shows histological results from these studies with depths.

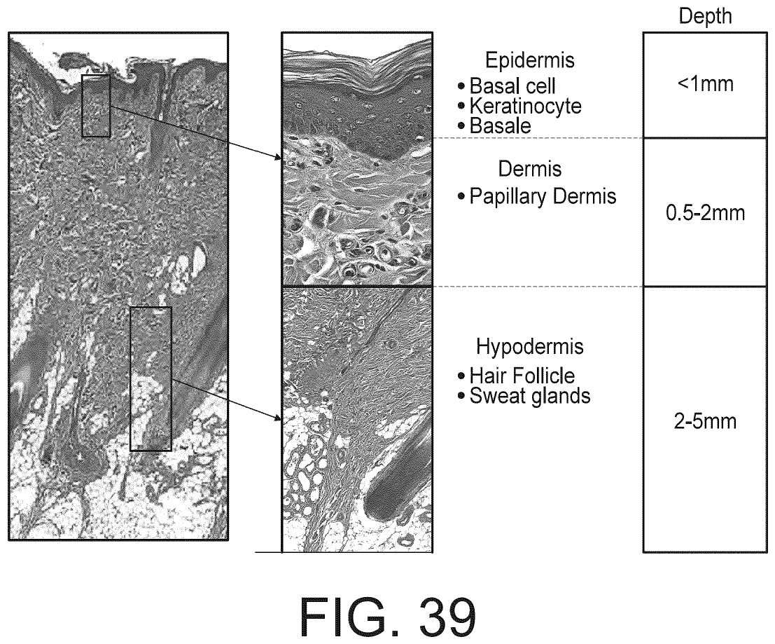

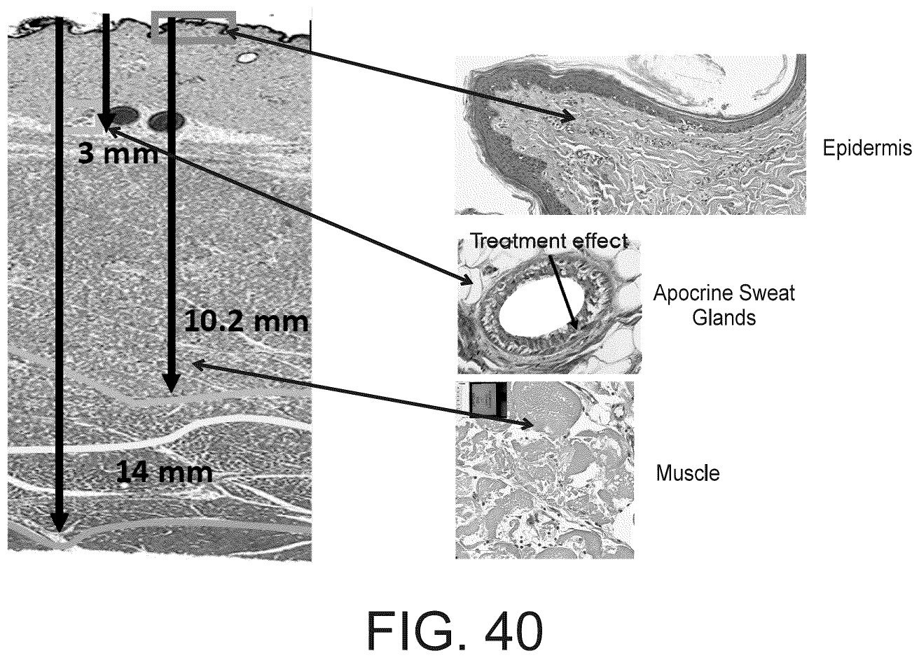

FIG. 39 illustrates cells in the dermal layer their approximate shapes and sizes and depths at which they exist including the epidermis layer the dermis layer and the hypodermis layer, and FIG. 40 illustrates histology from an animal experiment, in accordance with aspects of the disclosure. FIG. 40 shows treatment of the epidermis, the cells within, and the sweat glands at a tissue depth of approximately 3 mm, as well as treatment of muscle tissue at a depth of up to 14 mm. Each of these cell types in these images have been are effectively treated with the system described herein. FIGS. 39 and 40 illustrate the histology from an animal experiment using this invention and showing that it can treat the epidermis and the cells within, and the sweat glands at a tissue depth of approximately 3 mm and muscle tissue at a depth of up to 14 mm. This configuration was a prototype and within the mathematical bounds described herein, and one may tune the device and algorithm to address any and or all of these cell types at the various depths.

These results demonstrate the feasibility of the invention for the following indications; Benign lesions: Seborrheic keratosis, verruca, skin tags, viral warts, actinic keratoses, molluscum contagiosum, solar lentigo and hypertrophic/keloid scars, precancerous lesions, acne, sebaceous hyperplasia. In addition to treating sweat glands Hyperhidrosis, cancer, basal cell carcinoma, squamous cell and other dermatological indications discussed herein.

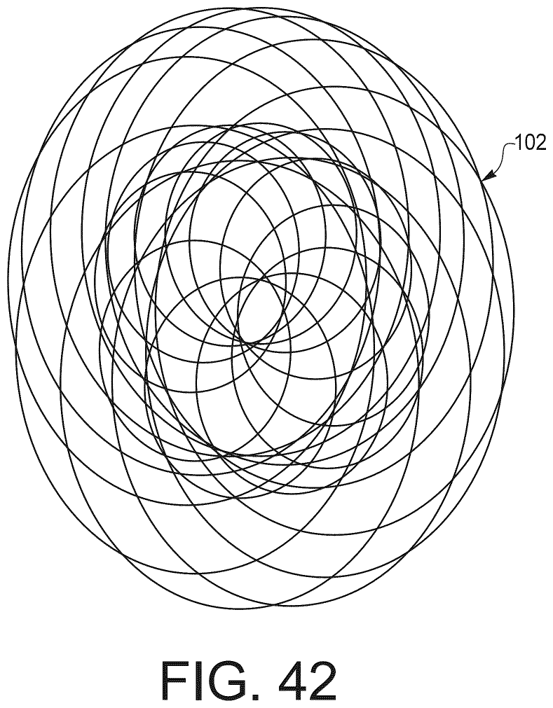

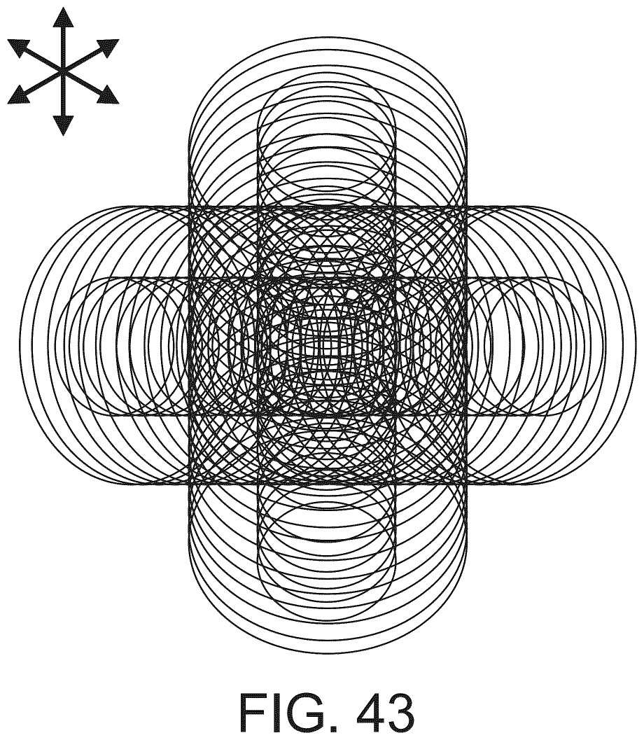

FIGS. 41-43 depict a surface location field treatment as an applicator traverses or scans over the surface of the tissue, in accordance with aspects of the disclosure. Based on the relative locations of the paths shown in FIGS. 41-43, the figures show that different patterns of movement of the applicator over the skin surface may provide different overall concentrations of energy, which may correspond to the particular treatment provided by the applicator. In accordance with some examples, by varying electrode geometry, size, and configuration, a treatment algorithm, and motion of the traversing or scanning by the applicator, tissue may be treated to depths of between about ½ mm to about 15 mm, without thermal damage to the tissues. For example, FIG. 41 shows that treatment with an applicator including ring electrodes resulting from moving the applicator left and right, offsetting the applicator up or down, and moving the application left and right, for six up and down offset positions. FIG. 42 shows treatment with an applicator including ring electrodes, which is moved in generally circular patterns. FIG. 43 shows treatment with an applicators including ring electrodes moved left and right, returned to a center position, and moved up and down. Darker lines in each of FIGS. 41-43 indicate increase treatment of certain areas relative to other treatments.

In accordance with some embodiments, the applicator may be a wireless applicator, which omits the cables and the generator. A wireless applicator may include, for example, one or more of the following:

A handle with a safety trigger switch and an arming thumb switch;
1″ inner diameter, 2″ outer diameter, coil of 0.24″ diameter coaxial wire in 2 layers, 0.1 m/turn for inner layer, 0.14 m/turn for outer layer, 4.2″ tall coil for 4.2 m (20 ns for each positive and negative pulse);
Two LiPo 18650 cells in series through the center of the coil (each is a cylinder 18.5 mm in diameter and 65.2 mm long, up to 100 A pulsed, 2.6 A hr., 3.7 Vea (Vnominal));
Dumbbell-like expansions on both ends of the handle;
Lower expansion containing electrodes, dummy load, contact roller, and tachometer, and spark gap;
Upper expansion contains controller PCB, HV supply, touch screen, charger contacts, and speaker;
Wall hanger/charger for storage;
Different units for different electrode areas or depths;
External screw for adjusting spark gap spacing, or to adjust the peak voltage; and/or
Removable batteries so that the remaining portion may be sterilized.

The disclosed systems and method provide numerous advantages. Monopolar current pulses may cause muscle twitch and pain sensation in living tissue. A simple capacitive discharge supply may generate an oscillating potential with an exponential envelop, but there may be a DC component to the average of the pulses.

Circuit design may produce pairs of nearly-square pulses separated by a small dwell time. Each bipolar pair may be separated by a pulse period, the inverse of the pulse frequency. The cost and weight of these pulsers make them less desirable for portable systems.

Stray fields from the electrodes may mobilize ions associated with the nearby nerve cells. Reversing the fields symmetrically within a time that is short compared to a nerve impulse duration (which is about 50 microseconds) may nearly eliminate nerve stimulation. A way to accomplish this for an isolated unipolar pulse generator is to allow the pulse exciting the electrodes to also excite a length of cable shorted at its end, such that the cable round-trip time is the required delay between the positive and negative pulses.

Delay lines, such as a Vvedensky pulse former, may superimpose a delayed inverted waveform on their output. The center conductor of a delay line of length La, such as a coax cable, may be shorted at one end to ground and attached at the other to an IRE electrode. The shield of the coax may be connected to a spark gap and to a bleed resistor from a high voltage supply. The signal velocity for the coaxial cable may be v_{coax}, giving a duration each pulse of L_{d}/v_{coax}, with no appreciable delay between the pulses, assuming the coaxial cable is impedance matched to the tissue sample. RG-316 cable may have a velocity of 4.7 ns/m, so a 5 m cable would produce sequential 23.5 ns pulses of opposite voltage at the IRE electrodes. 5 m of RG-316 weighs 0.18 lb., and fills 32 cc of volume. The spark gap may determine the peak voltage experienced by the sample. The impedance of the bleed resistor may be large compared to the cable impedance. The cable may act as the HV storage capacitor. The dummy load in parallel with the IRE electrodes may have an impedance larger than the cable and the expected sample impedance; such that the dummy load may act as a safety energy dump if inadequate contact is being made between the electrodes and the tissue sample.

The delay line used may impedance-matched to the cable. Matched loads at the generator may suppress reflections. In such instances, an initial charging voltage may be twice the desired voltage of either half of the resulting bipolar pulse. Further the positive and negative pulses may arrive with no delay in between, unless switches are added to the circuit. Advantages may include the charging voltage need not be pulsed; and it may be provided by a DC supply with a current-limiting resistor. Additionally a high voltage capacitor may be omitted, as the cable may act as the capacitor. The length of the cable may be changed to control timing—e.g., for 30 ns for the combined pair, the equation of 30 ns/(4.7 ns/ft*2)=3.2 m cable length for RG-316 coaxial cable. The total energy delivered may be limited by

$$0.5 ⋆ {V}_{max}^{2} ⋆ C ,$$

where C is the cable capacitance.

Whether electrolytic gel, saline, semiconductive film, or filled polymer, the conductive medium may be at least as conductive than tissue and less conductive than metal. Their application to the surface of the tissue being treated may be omitted, but the conductive medium may facilitate both distribution of electric field and the reduction of arc formation. The gel may be applied using different methods. One method is to first apply the gel to the skin, and then subsequently scan the skin surface by passing the electrodes across the skin, which may also coat the electrodes and their mounting structure, while also filling gaps between the skin, electrodes, and mounting. Another method may include using channels and passages to apply gel to the skin surface while moving the electrodes across the skin. This applies the gel to the area being scanned, and if the back pressure of extruding the gel is monitored, a signal indicating good contact between the holder and the skin may be an input to the controller. The conductive material may include a hydrogel with a metal component infused into it, or a hydrogel with a salt base. The use of gel may be minimized by shaving the dermis, roughening up the dermis, chemically treating the dermis or another method of modifying the stratum corneum such that the contact to the tissue is more direct. As the voltages increase gel may be used to suppress arcing.

Sufficient electric field intensity and duration to reliably porate 3-5 micron cell membranes may be incorporated into a system or apparatus used in IRE treatment. As the diameter of a cell membrane shrinks, the magnitude of the externally applied electric field may increase to obtain the requisite potential difference across the membrane for rapid and permanent poration. For example, a typical 30 micron diameter cell might reach this level of poration for a 3 KV/cm field applied for about 1 msec. Higher fields may be applied for shorter periods of time, to constrain ohmic heating to a less than about a few degrees C. The lethal electroporation threshold (LET) has been demonstrated resulting from 35 KV/cm fields for 1 us, and for single 20 nsec, 55 KV/cm pulses. These short intense pulses result in lower heating of the tissue, thereby avoiding thermal damage.

An analogy to electroporation of cells is standard voltage breakdown of a capacitor. The AC voltage required for capacitor breakdown tends to be lower than such a DC voltage. The time-to-breakdown is a non-linear function based on the size of the over-voltage. There are thermal effects that amplify the originating arc breakdown, which also amplify the resulting damage. Thus, in some aspects of the disclosure, the treatment incorporates higher electric fields and lower pulses, using the non-linearity of the response, to reduce the deposited energy density in the tissue.

Membranes of cells larger than bacteria in the near-surface high-field treatment regions may tend to see at least as much membrane poration as the bacteria, so they are at risk of cell death. To minimize the number of normal tissue cells affected, the IRE treatment may provide a shallow treatment depth, while still treating the surface-deposited bacteria. The treatment enables the treated volume to experience a temperature rise of less than about 4 deg C., with minimized collateral thermal damage of healthy tissue. As discussed, the treatment may limit the high electric field to the surface 0.3 mm of the skin.

A simple type of high voltage electrical pulse is the discharge of a capacitor through a spark gap, resulting in a fast linear rise and an exponential decay. With respect to timing of a treatment electrical pulse, a pulse is fired when the tissue is present, and when it has moved about 0.3 mm perpendicularly with respect to the axes of the two electrodes, up to rates of about 600 Hz. The single positive-going-voltage exponential decay may be followed by a single negative-going-voltage exponential decay.

Current conduction is may only take a few nanoseconds to get enough ions gathered on surfaces to collapse the field onto insulators blocking the current flow. When there are parallel paths that are not insulated, these paths may be supplied with enough current to maintain the local field strength.

The current is low in the embodiments that include two cylindrical electrodes because the contact area with the skin of the patient is low. The low contact area increases the resistance, which reduces the current of the electric field applied to the skin and the tissue of the patient. The power is low in the embodiments that include two cylindrical electrodes because the tissue treatment volume is low (small area*small depth) and the temperature rise is low (<1 deg C.).

In some embodiments, the smallest organelles to be affected are 10 microns/KV/cm.

In some embodiments, the minimum effective pulse width is set by the sodium ion mobility within the cells and the strength of the electric field.

With respect to variable affecting the above-discussed calculations, the following is submitted. In electrical terminology, the membrane functions as a combined resistor and capacitor. Resistance arises from the fact that the membrane impedes the movement of charges across it. Capacitance arises from the fact that the lipid bilayer is relatively thin, such that an accumulation of charged particles on one side gives rise to an electrical force that pulls oppositely charged particles toward the other side. The capacitance of the membrane is relatively unaffected by the molecules that are embedded in it, so the membrane has a more or less invariant value estimated at 2 μF/cm² (the total capacitance of a patch of membrane is proportional to its area). The conductance of a pure lipid bilayer is so low that in biological situations it is always dominated by the conductance of alternative pathways provided by embedded molecules. Thus, the capacitance of the membrane is more or less fixed, although the resistance is highly variable.

As stated above, the thickness of a membrane is estimated to be about 7 nm to about 8 nm. Because the membrane is so thin, the membrane does not take a very large transmembrane voltage to create a strong electric field within the membrane. Typical membrane potentials in animal cells are on the order of 100 millivolts (that is, one tenth of a volt), but calculations show that this generates an electric field close to the maximum that the membrane may sustain. It has been calculated that a voltage difference much larger than 200 millivolts could cause dielectric breakdown, that is, arcing across the membrane.

Regulators of cell excitability include the extracellular electrolyte concentrations (i.e. Na+, K+, Ca2+, Cl−, Mg2+) and associated proteins. Proteins that regulate cell excitability are voltage-gated ion channels, ion transporters (e.g. Na+/K+-ATPase, magnesium transporters, acid-base transporters), membrane receptors and hyperpolarization-activated cyclic-nucleotide-gated channels. For example, potassium channels and calcium-sensing receptors are regulators of excitability in neurons, cardiac myocytes and many other excitable cells like astrocytes. Calcium ion is also a messenger in excitable cell signaling. Activation of synaptic receptors initiates long-lasting changes in neuronal excitability. Thyroid, adrenal, and other hormones also regulate cell excitability, for example, progesterone and estrogen modulate myometrial smooth muscle cell excitability.

Many cell types are considered to have an excitable membrane. Excitable cells include neurons, muscle (cardiac, skeletal, smooth), vascular endothelial cells, pericytes, juxtaglomerular cells, interstitial cells of Cajal, many types of epithelial cells (e.g., beta cells, alpha cells, delta cells, enteroendocrine cells, pulmonary neuroendocrine cells, pinealocytes), glial cells (e.g., astrocytes), mechanoreceptor cells (e.g., hair cells and Merkel cells), chemoreceptor cells (e.g. glomus cells, taste receptors), some plant cells, and some immune cells. Astrocytes display a form of non-electrical excitability based on intracellular calcium variations related to the expression of several receptors through which they may detect the synaptic signal. In neurons, there are different membrane properties in some portions of the cell, for example, dendritic excitability that endow neurons with the capacity for coincidence detection of spatially separated inputs.

The temperature rise required for effective IRE treatment may be a function of organelle size. This temperature also may depend on the cell wall composition. The lipid bilayer, made up of two layers of phospholipids with cholesterols (a lipid component) interspersed between them, maintains appropriate membrane fluidity at various temperatures. The cell membrane includes three classes of amphipathic lipids: phospholipids, glycolipids, and sterols. The amount of each lipid depends upon the type of cell, but in the majority of cases phospholipids are the most abundant, often contributing for over 50% of all lipids in plasma membranes. Cholesterol is normally found dispersed in varying degrees throughout cell membranes, in the irregular spaces between the hydrophobic tails of the membrane lipids, where it confers a stiffening and strengthening effect on the membrane. Additionally, the amount of cholesterol in biological membranes varies between organisms, cell types, and even in individual cells. Cholesterol, a major component of plasma membranes, regulates the fluidity of the overall membrane, meaning that cholesterol controls the amount of movement of the various cell membrane components based on its concentrations. At high temperatures, cholesterol inhibits the movement of phospholipid fatty acid chains, causing a reduced permeability to small molecules and reduced membrane fluidity. The opposite is true for the role of cholesterol in cooler temperatures. Cholesterol production, and thus concentration, is up-regulated (increased) in response to cold temperature. At cold temperatures, cholesterol interferes with fatty acid chain interactions. Acting as antifreeze, cholesterol maintains the fluidity of the membrane. Cholesterol is more abundant in cold-weather animals than warm-weather animals. In plants, which lack cholesterol, related compounds called sterols perform a similar function as cholesterol.

Proteins content of the cells also may affect outcomes. The cell membrane has large content of proteins, typically around 50% of membrane volume. These proteins are responsible for various biological activities. Approximately a third of the genes in yeast code specifically for them, and this number is even higher in multicellular organisms. Membrane proteins include three main types: integral proteins, peripheral proteins, and lipid-anchored proteins.

For the prokaryotic membranes, there are multiple factors that may affect the fluidity. One factor that may affect the fluidity is fatty acid composition. For example, when the bacteria Staphylococcus aureus was grown in 37 deg C. for 24 h, the membrane exhibited a more fluid state instead of a gel-like state. This supports the concept that in higher temperatures, the membrane is more fluid than it is in colder temperatures. When the membrane is becoming more fluid and needs to become more stabilized, it may make longer fatty acid chains or saturated fatty acid chains in order to help stabilize the membrane

The disclosed systems and methods provide for tissue surface bactericide. The disclosed IRE treatment has been reduced to practice in several tissue types demonstrating effectiveness on cells in each of the categories described herein. Cell types representing those that may be addressed for conditions such as, excessive sweating (hyperhidrosis), acne (sebaceous hyperplasia), cancer (BCC & SCC), chronic wound neutrophils, and bacteria.

Researchers have been involved in over 84 porcine animal studies and hundreds of bench top studies refining IRE to induce cell lysis absent of thermal damage and macrophage proliferation (e.g., minimal Inflammation) which indicates lysis through apoptotic cascades.

Therapy modelling with various electrode designs, and algorithms were confirmed with histological results. Demonstrated histological effects from 5000 V electrode design @ 5000 V for 8500 V/cm: about 0.2 mm depth, small epidermal cells; 2500 V/cm: ˜7 mm depth, large cells, sweat glands; and 1000 V/cm: about 14 mm depth, skeletal muscle cells. Histologic assessment showed the following: for the epidermis: epidermal effect for the apocrine gland: foci consistent with IRE changes, 3 mm; for skeletal muscle: changes demonstrate gradient effect and extend to side and base of tissue section; upper region consistent with IRE changes, at least 10.2 mm; upper middle region combination of consistent with IRE changes, at least 12 mm; Lower Middle Region changes consistent with IRE.

Sanitizing the surface of a wound or of skin may be complicated because bacteria form islands that are protected by a surface layer of dead bacteria. That protective surface layer may tend to isolate the active bacteria from chemical or UV treatment. Abrading the skin or wound surface may remove this layer of protection, but abrading also damaging the surface tissue. In contrast, the disclosed IRE treatment may kill the bacteria within about 100 um of the skin surface, without damaging underlying tissue and without heating the surface by more than 0.1 deg C.

These disclosed IRE treatment may provide an electroporation approach for sterilizing skin-surface bacteria. As discussed, the target sterilization depth may be limited to about 0.3 mm, with minimal or no tissue damage. The IRE treatment maty be effective against most bacteria types, including antibiotic resistant strains. The IRE treatment is easy to apply and inexpensive.

Electroporated cell membranes allow transport of surrounding material into the cell, and enhances efficacy of other antibacterial treatments, such as suspended silver nanoparticles, antibiotics, and other interventions.

When an optimal IRE treatment frequency for ‘normal’ 30 micron cells are about 1,000 ns pulses, the optimum treatment for affecting 5 micron bacteria is about 167 ns, and an optimum treatment for affecting 0.3 micron bacterial may be 10 ns. Using this estimate as a guide, the 20 ns single pulse treatment at 55 KV may be used to kill bacteria.

The following is presented regarding nerves and neuromuscular junction effects. Application of the disclosed IRE or pulsed electric field (PEF) to nerves results in the nerve function being altered. The amount and profile or type of energy applied may result in either short term or long term alteration. The energy may be tailored to any traditional nerve block locations and technics. The electrodes may be configured to be deployed via a cannula, needle, or catheter, similar to existing procedural techniques. Additionally large near surface epidermal areas may be treated with electrodes treating the surface receptors on skin. The nerve may be temporarily affected by this treatment as to stop the signal transfer for a period of time. The nerve cell may either be permanently disabled such that it will have to grow back, or temporarily disabled such that it has to repair itself. The energy may not destroy the tissue scaffold and therefore may allow the rapid healing of the nerve. The treatment may affect both myelinated and non-myelinated nerves depending on the voltage applied. The neuromuscular junction anatomy may also be affected by IRE, PEF, or pulsed field ablation (PFA). When the neuromuscular junction is treated, the muscle response is disabled for a period of time while the tissue remodel and heal. This has been demonstrated clinically. The effects of PFA on the neuromuscular junction include temporarily deactivation the function and signaling mechanisms. PFA is a nonthermal method of tissue ablation that utilizes high-amplitude pulsed electric fields to create irreversible electroporation (IRE) in cells. In contrast to traditional thermal ablation methods like radiofrequency ablation, PFA is not intended to destroy nerves. In fact, PFA is sometimes considered safer for nerves than thermal ablation. The neuromuscular junction (NMJ) may be affected as follows:

### 1. Nerve Stimulation and Temporary Stunning Observed

PFA pulses may excite nerve tissues at lower electric field strengths than those required for irreversible damage to cardiomyocytes. In some cases, PFA may cause transient stunning or temporary dysfunction of nerves, like the phrenic nerve, which is involved in breathing. While these effects are usually temporary and nerve function recovers, their occurrence may depend on factors such as PFA intensity and catheter proximity to the nerves.

### 2. Potential Mechanisms for Transient Nerve Dysfunction

The electric fields may induce hyperpolarization of nerve cells, leading to a temporary inability to transmit action potentials. Another theory proposes that the electric fields could disrupt calcium ion flow at the motor endplate, reducing acetylcholine release and preventing effective muscle activation. The nerve cells themselves may be destroyed eliminating the process completely

### 3. Preservation of Nerve and Muscle Structure

PFA may spare the structural integrity of nerves and surrounding tissues, including the endoneurium, epineurium, and perineurium, which may facilitate axonal regeneration and potential functional recovery. Some research suggests that PFA causes minimal impact on nerve function when applied to cardiac tissue and that any functional impairment observed may recover over time. PFA may lead to transient stimulation and potential stunning of nerves and muscles at the neuromuscular junction, it appears to preserve the structural integrity of these tissues, offering potential advantages over thermal ablation.

Examples include the following.

The disclosed IRE treatment may be used as follows. Genicular nerve block may be used to treat the symptoms of chronic knee pain from osteoarthritis, failed knee replacement, patients medically unstable for knee replacements, and patients who want to avoid knee replacements.

The nerve blocks may inhibit impulse transmission distally in a nerve terminal, thus terminating the pain signal perceived by the cortex. Nerve blocks may be used to treat acute pain (e.g., procedural anesthesia and perioperative analgesia), as well as for diagnosis and treatment of chronic pain. Impulse blockade may be brief (e.g., hours) or prolonged (e.g., months), depending on the dosage or energy used in the block and the location on the nerve. Nerve blocks are also useful in the emergency department for acute pain management of the extremities. Other uses for IRE treatment include:
Anesthesia of the extremity for procedures;
Alternative to opioids in certain patient populations (e.g., a head injury patient, patients with concomitant mental status change, patients on buprenorphine);
Various nerves may be blocked depending on the injury. These include the following upper or lower extremities;
Brachial plexus roots at the interscalene location block the shoulder, upper arm, elbow, and forearm;
Brachial plexus trunks at the supraclavicular location block the upper arm, elbow, wrist, and hand;
Brachial plexus cords at the infraclavicular location block the upper arm, elbow, wrist, and hand;
Brachial plexus branches at the axillary location block the forearm, wrist, hand, and elbow, including the musculocutaneous nerve;
The median nerve at the elbow blocks the hand and forearm;
The radial nerve at the elbow blocks the hand and forearm;
The ulnar nerve at the elbow blocks the hand and forearm;
The femoral nerve at the femoral crease blocks the anterior thigh, femur, knee, and skin anesthesia over the medial aspect of the leg below the knee;
The sciatic nerve at the subgluteal location or anterior approach below the femoral crease blocks the posterior aspect of the thigh and the anterior, lateral, and posterior lower leg, ankle, and foot;
The sciatic nerve at the popliteal location blocks the anterior, lateral, and posterior lower leg, ankle, and foot; or
Ankle block of five separate nerves to the ankle and foot (saphenous nerve, deep peroneal nerve, superficial peroneal nerve, posterior tibial nerve, and sural nerve) blocks the entire foot.

Nerve blocks offer many advantages compared to traditional anesthetic and analgesic techniques. Patients who would otherwise have excessive risks with general anesthesia may safely undergo surgery painlessly with IRE treatment. Additionally, the adverse effects of perioperative opioid analgesia may be minimized or avoided entirely while still providing superior pain control.

Another example is peripheral neuropathy. Peripheral neuropathy is a combination of conditions and treatments described herein, and IRE may facilitate relief in these patients by eliminating the pain signaling and helping foster circulation within the affected tissue or organ. The sarcolemma in muscle cells may also undergo IRE treatment. The sarcolemma transmits synaptic signals, generates action potentials, and is involved in muscle contraction. Unlike other cell membranes, the sarcolemma makes up small channels called T-tubules that pass through the entirety of muscle cells. The average sarcolemma is 10 nm thick as opposed to the 4 nm thickness of a general cell membrane.

IRE treatment may be used for treatment of facial wrinkles currently mainly treated with Botox. The disabling of the neuromuscular junction in the face by IRE treatment may facilitate the muscle relaxation response.

To further increase effectiveness of pulsed electric field may be used in combination with other energy forms to increase, accelerate, and activate the healing response, as follows:
1. IRE and Ultrasonic waves;
2. IRE and Heat;
3. IRE and Chemicals;
4. IRE and Shock waves;
5. IRE and Microwave;
6. IRE and Cryotherapy;
7. IRE and Physical distortion, stretching or compression.;
8. IRE and Vibration;
9. IRE with Suction (e.g., negative pressure);
10. IRE with Barotrauma is physical damage over-stretching the tissues in tension or shear, either directly by an expansion of the gas in the closed space or by pressure difference hydrostatically transmitted through the tissue.

These and other methods may be combined to facilitate better responses to therapeutic treatments.

In accordance with some aspects of the disclosure, the systems and methods may include the following items:

Item 1: An electroporation system comprising a non-penetrating irreversible electroporation (IRE) electrode that is configured to treat tissue to a depth of more than 15 mm.

Item 2: The system of item 1, wherein the electrodes comprises at least one of an approximately hemi-cylindrical, or hemi-elliptical electrode.

Item 3: The system of item 1, further comprising at least a second electrode, wherein a treatment depth corresponds to a distance between the electrodes.

Item 4: The system of item 1, further comprising at least a second electrode, wherein the first electrode receives a positive voltage, and the second electrode receives a negative voltage.

Item 5: The system of item 1, wherein a treatment field strength is determined based on is a size of a cell or organelle to be treated.

Item 6: An electroporation system comprising a plurality of irreversible electroporation (IRE) electrodes, each having a maximum electric field strength E0 proximate to a majority of the electrode surfaces, wherein the electrodes do not emit a treatment field strength of more than 0.3*E0, where E0 is a potential difference between one of the electrodes and bulk tissue divided by a local radius of curvature of a surface of the electrode contacting the tissue.

Item 7: The system of item 6, wherein the at least one of the electrodes comprises a cylindrical electrodes applied tangent to a treatment surface, wherein conductive gel fills adjacent void between the treatment surface and the electrodes.

Item 8: The system of item 6, wherein a gap between the electrodes and an insulating support is filled with a semi-conductive material to attenuate an electric field gradient experienced by the surface of the tissue.

Item 9: The system of item 8, wherein the semi-conductive material comprises a polymer with conductive particles.

Item 10: The system of item 8, wherein the semi-conductive material is an electrolyte gel.

Item 11: An electroporation system configured to move in a direction tangent to a treatment surface during treatment.

Item 12: The system of item 11, wherein the treatment is configured to raises a temperature of the treated volume by less than 3 deg C.

Item 13: The system of item 11, wherein the system is configured to treat an epidermis of a patient as the treatment surface, without cutting or abrading a stratum corneum of the patient.

Item 14: The system of item 13, wherein the system is configured to apply a field gradient sufficient to temporarily form electrical discharge channels through the stratum corneum.

Item 15: The system of item 13, wherein the system is configured to apply a field that is bipolar or biphasic, thereby to minimize nerve participation.

Item 16: The system of item 15, wherein the system comprises a coaxial pulse former configured to generate a bipolar field.

Item 17: The system of item 11, wherein a peak electrical power applied to the treatment surface is less than 10 Kw.

Item 18: The system of item 11, wherein the system comprises electrodes, wherein the system is configured to applies a conductive gel between the electrodes and the treatment surface during movement of the electrodes across the treatment surface.

Item 19: The system of item 11, wherein the system comprises a portable handheld wand.

Item 20: An irreversible electroporation (IRE) system configured to treat an epidermis of a patient without cutting or abrading the stratum corneum.

Item 21: The system of item 20, wherein an applied field gradient of an electrical field applied by the system is sufficient to temporarily form electrical discharge channels through a stratum corneum of the epidermis of the patient.

Item 22: The system of item 20, wherein the system is configured to apply a field that is bipolar or biphasic, thereby to minimize nerve participation.

Item 23: An irreversible electroporation (IRE) system comprising electrodes configured to apply an electric field to biological tissue adjacent the tissue while the electrodes are moving with respect to the tissue.

Item 24: The system of item 23, further comprising a sensor configured to time an application of the electric field based on the movement of the electrodes, thereby to achieve a target treatment aerial uniformity.

Item 25: The system of item 23, wherein the system is configured to apply a conductive gel between the electrodes and the tissue while the electrodes are moving.

Item 26: A bipolar irreversible electroporation (IRE) system comprising a monopolar voltage pulser with a delay line, configured to form a bipolar pulse pair with a delay.

Item 27: An irreversible electroporation (IRE) system comprising a plurality of cylindrical electrodes configured to be applied tangent to a treatment surface, and configured to fill a void between the surface and the cylindrical electrodes with a gel.

Item 28: A tissue surface irreversible electroporation (IRE) system comprising two locally parallel electrodes, wherein a spacing between the electrodes is determined such that an electrical breakdown distance in air at an operating voltage is exceed, and wherein a diameter of the electrodes is determined to provide a predetermined desired electric field decay length into the treatment surface.

Item 29: A tissue surface nonpenetrating irreversible electroporation (IRE) system, wherein the system if configured such that a deposition of aerial energy density results in a temperature rise of less than 2 deg C. in treated tissue.

Item 30: A tissue surface nonpenetrating irreversible electroporation (IRE) system, wherein the system comprises a portable handheld wand.

Item 31: A tissue surface nonpenetrating irreversible electroporation (IRE) system, wherein the system is configured such that pulse amplitudes and duty cycles provide a treatment with an energy densities of 50,000 V/cm at a depth of less than 1 mm.

Item 32: A tissue surface nonpenetrating irreversible electroporation (IRE) system, wherein the system is configured such that pulse amplitudes and duty cycles provide a treatment with an energy densities of 5,000 V/cm at a depth of less than 5 mm.

Item 33: A tissue surface nonpenetrating irreversible electroporation (IRE) system, wherein the system is configured such that pulse amplitudes and duty cycles provide a treatment with an energy densities of 1,000 V/cm to 2500 V/cm at a depth of less than 14 mm.

Item 34: A tissue surface nonpenetrating irreversible electroporation (IRE) system, wherein the system is configured to provide a treatment with a current draw of 5 A to 200 A.

Item 35: A tissue surface nonpenetrating irreversible electroporation (IRE) system, wherein the system is configured such that pulse amplitudes and duty cycles provide a treatment for a surface area of 1 square inch to 10 square inches.

Item 36: A tissue surface nonpenetrating irreversible electroporation (IRE) system, wherein the system is configured such that pulse amplitudes and duty cycles are modified based on sensor inputs for at least one of motion, thermal change, impedance change, current draw, or voltage.

Item 37: A nonpenetrating irreversible electroporation (IRE) system, wherein the system is configured to be applied to an epidermis to selectively modify responsivity of nerve cells near a neuromuscular junction.

Item 38: A nonpenetrating irreversible electroporation (IRE) system, wherein the system is configured to sterilize near-surface bacteriological infection.

Item 39: A tissue surface nonpenetrating irreversible electroporation (IRE) system, wherein the system is configured such that pulse amplitudes and duty cycles are determined to provide at least one of at least one of pain reduction, hyperhidrosis, neutrophils and bacteria removal, seborrheic keratosis removal, actinic keratosis removal, molluscum contagiosum removal, solar lentigo and hypertrophic/keloid scar removal, precancerous lesion removal, acne removal, or sebaceous hyperplasia removal.

Item 40: A tissue surface nonpenetrating irreversible electroporation (IRE) system, wherein the system is configured such that pulse amplitudes and duty cycles are determined to treat at least one of a cell, fibroblast, bacteria, neuromuscular junction, muscles cell, sweat gland, basal cell, keratinocyte, hair follicle, papillary cell, nerve cell, seborrheic keratosis, actinic keratosis, molluscum contagiosum, solar lentigo and hypertrophic/keloid scar, precancerous lesion, acne, sebaceous hyperplasia, organelle, apocrine gland, melanocyte, dendritic cell, merkel cell, stratum granulosum, stratum lucidum, stratum corneum, or nerve block.

Item 41: A tissue surface nonpenetrating irreversible electroporation (IRE) system, wherein the system is configured such that treatment does not penetrate into tissue, and treatment treats dermatology indications including hyperplasia.

Item 42: A tissue surface nonpenetrating irreversible electroporation (IRE) system, wherein the system is configured such that pulse amplitudes and duty cycles are treat cells of a variety of certain sizes, shapes, and morphology at various depths into an epidermis, dermis, and hypodermis.

Item 43: A tissue surface nonpenetrating irreversible electroporation (IRE) system, wherein the system is configured such that pulse amplitudes and duty cycles are determined to treat at least one of a virus, mycoplasma, bacteria, yeast, eukaryotic, or mycelia.

Item 44: A tissue surface nonpenetrating irreversible electroporation (IRE) system, wherein the system is configured such that pulse amplitudes and duty cycles are determined to treat cells in a range from 0.05 um to greater than 500 um.

Item 45: A tissue surface nonpenetrating irreversible electroporation (IRE) system, wherein the system is configured such that pulse amplitudes and duty cycles are determined to treat cells with pulses ranging from 1 pulse to 500 pulses per cell.

Item 46: A tissue surface nonpenetrating irreversible electroporation (IRE) system, wherein the system is configured such that pulse amplitudes and duty cycles are determined to result in a temperature rise of less than 2 deg C. in treated tissue.

Item 47: A tissue surface nonpenetrating irreversible electroporation (IRE) system, wherein the system is configured such that pulse amplitudes and duty cycles are determined to treat cells with a single pulse having an electric field strength of 50 KV/cm.

Item 48: A tissue surface nonpenetrating irreversible electroporation (IRE) system, wherein the system is configured such that pulse amplitudes and duty cycles are determined to treat at least one of cells for non-permanent nerve disruption for temporary pain relief, acute surgical pain relief, chronic pain relief, osteoarthritic pain relief, or diabetic pain relief.

Item 49: A tissue surface nonpenetrating irreversible electroporation (IRE) system, wherein the system is configured such that pulse amplitudes and duty cycles are determined to treat cells without creating scar tissue.

Item 50: A tissue surface nonpenetrating irreversible electroporation (IRE) system, wherein the system is configured such that pulse amplitudes and duty cycles are determined to treat cells for non-permanent nerve disruption for skin rejuvenation.

Item 51: A tissue surface nonpenetrating irreversible electroporation (IRE) system, wherein the system is configured such that pulse amplitudes and duty cycles treat cells to kill excessive neutrophils and prevent release of mediators signaling neutrophils production.

Item 52: A tissue surface nonpenetrating irreversible electroporation (IRE) system, wherein the system is configured such that pulse amplitudes and duty cycles treat cells, wherein the system does not include a cooling system.

Item 53: A tissue surface nonpenetrating irreversible electroporation (IRE) system, wherein the system is configured such that pulse amplitudes and duty cycles treat cells with an electrode configured either drop local field intensity or focus field intensity.

Item 54: A tissue surface nonpenetrating irreversible electroporation (IRE) system, comprising a plurality of electrodes, wherein the system is configured such that pulse amplitudes and duty cycles treat cells, wherein a spacing between the electrodes is determined to provide a uniform field at a predetermined depth.

Item 55: A tissue surface nonpenetrating irreversible electroporation (IRE) system, wherein the system is configured such that pulse amplitudes and duty cycles treat cells to reduces wrinkles by temporarily paralyzing muscles or by blocking the nerve signals ordering muscles to contract, thereby resulting in muscle relaxation and a smoothing of skin overlying the muscles.

Item 56: A tissue surface nonpenetrating irreversible electroporation (IRE) system, wherein the system is configured such that pulse amplitudes and duty cycles treat cells to treat diabetic peripheral neuropathy by temporarily affecting nerves.

Item 57: A tissue surface nonpenetrating irreversible electroporation (IRE) system, wherein the system is configured such that pulse amplitudes and duty cycles treat cells while preserving an extracellular matrix but causing cell lysis.

Item 58: A surface scanning depth controlled nonpenetrating irreversible electroporation (IRE) system, wherein the system is configured to kill bacteria.

Item 59: A tissue surface nonpenetrating irreversible electroporation (IRE) system, comprising a pulse-inverting delay line.

Item 60: A wireless tissue surface nonpenetrating irreversible electroporation (IRE) system, wherein the system is a portable wand configured to provide dermatology procedures.

Item 61: A two dimensional nonpenetrating irreversible electroporation (IRE) electrode array, where electrode array is configured to suppress air breakdown.

Item 62: A variable thickness resistive coating on nonpenetrating irreversible electroporation (IRE) electrode, wherein the coating is configured to control edge-effect hot spots.

Item 63: A method of treatment, comprising: applying electrical energy to a skin surface with a non-penetrating electrode, wherein the electrical energy generates a field in tissue underlying the skin surface and causes lysis to a cell or vesicle under the skin surface.

Item 64: The method of item 63, wherein the applying electrical energy comprises delivering a series of bipolar electric pulses, wherein there is little to no delay of between delivery of positive polarity and negative polarity electric pulses.

Item 65: The method of item 63, wherein the applied electrical energy does not damage an extracellular matrix of the patient.

Item 66: The method of item 63, wherein the applying electrical energy comprises applying an electric field with a field strength of about 1,500 V/cm to about 50,000 V/cm.

Item 67: The method of item 63, wherein the applying electrical energy comprises applying an electric field having a field strength of greater than about 50,000 V/cm.

Item 68: The method of item 63, wherein the applying electrical energy comprises applying 1 to 10 pulses with an electric field having a field strength of about 30,000 V/cm to about 55,000 V/cm, to causes lysis to a depth of up to about 1 mm.

Item 69: The method of item 63, wherein the applying electrical energy comprises applying 100 to 1000 pulses with an electric field having a field strength of about 1500 V/cm to about 7000 V/cm, to causes lysis to a depth of up to about 15 mm.

Item 70: The method of item 63, wherein the applying electrical energy comprises applying an electric field having a frequency of about 100 Hz to about 1 MHz

Item 71: The method of item 63, wherein the applying electrical energy comprises applying a series of bipolar pulses of about 0.5 KV to about 50 KV.

Item 72: The method of item 63, wherein the applying electrical energy comprises applying a series of pulses, wherein each of the pulses has a duration of about 0.5 ns to about 1000 us.

Item 73: The method of item 63, further comprising determining, prior to the applying electrical energy, an amount of energy to be applied to the skin surface based on a type of the tissue underlying the skin.

Item 74: The method of item 63, further comprising determining, prior to the applying electrical energy, an amount of energy to be applied to the skin surface based on a size of a target cell.

Item 75: The method of item 63, further comprising determining, prior to the applying electrical energy, an amount of energy to be applied to the skin surface based on an average spacing between membranes in the tissue underlying the skin.

Item 76: The method of item 63, further comprising determining, prior to the applying electrical energy, an amount of energy to be applied to the skin surface based on a voltage difference to be generated in the tissue underlying the skin.

Item 77: The method of item 63, wherein the applying electrical energy does not generate a rise in temperature that destroys cells at the skin surface.

Item 78: The method of item 63, wherein the applying electrical energy does not cause a muscle or nerve twitch response.

Item 79: A tissue surface electroporation system whose pulse amplitudes and duty cycles provides a means for treatment of small-organelle membrane destruction to kill surface bacterial colonies.

Item 80: A non-penetrating electroporation electrodes that treat tissue to a depth of more than 15 mm,
where the electrodes are approximately hemi-cylindrical, or hemi-elliptical;
where the treatment depth is on the order of the spacing of the electrodes;
where the sign of the applied potential alternates between nearest neighbor electrodes;
where bipolar excitation minimizes nerve involvement; and/or
where the treatment field strength is selected from the size of the cells or organelles to be treated.

Item 81: Electroporation electrodes that have a maximum electric field strength E0 proximate to the majority of the electrode surfaces, and that nowhere project a treatment field strength more than 0.3*E0, thereby insuring non-thermal treatment;
where E0 is the potential difference of the conductive electrode to the bulk tissue divided by the local radius of curvature of the electrode surface contacting the tissue;
where cylindrical electrodes applied tangent to the treatment surface, with conductive gel filling the adjacent void between the surface and the cylinders;
where a gap between the electrodes and insulating support is filled with semi-conductive material to attenuate the electric field gradient experienced by the surface of the tissue;
where the semi-conductive material is a polymer loaded with conductive particles;
where the semi-conductive material is an electrolyte gel; and/or
where the aerial energy density deposited insures a local temperature rise in the tissue of less than 0.3 deg C.

Item 82: An electroporation system that moves tangent to a treatment surface during treatment;
where treatment raises the treated volume temperature by less than 3 deg C.;
where the epidermis is treated without cutting or abrading the stratum corneum;
where the applied field gradient is sufficient to temporarily form electrical discharge channels through the stratum corneum;
where the applied field is bipolar or biphasic to minimize nerve participation;
which incorporates a coax pulse former to generate bipolar fields;
where the peak electrical power applied is less than 10 Kwatts;
where the system applies a conductive gel between the electrodes and the tissue as the electrodes are moving; and/or
where the system is entirely contained in a portable handheld wand.

It will be apparent to those skilled in the art that various modifications and variations can be made to the disclosed system and method without departing from the scope of the disclosure. Other embodiments of the system and method will be apparent to those skilled in the art from consideration of the specification and practice of the apparatus and system disclosed herein. It is intended that the specification and examples be considered as exemplary only, with a true scope of the disclosure being indicated by the following claims and their equivalents.

1. A system for performing irreversible electroporation within a tissue under a skin surface of a patient, the system comprising:
a power source configured to generate power;
a base; and
at least one electrode extending from the base, wherein the at least one electrode is configured to receive the power and based on the power to generate an electric field sufficient to porate a cell membrane within the tissue while increasing a temperature of the skin surface less than 3 deg C. during poration.

2. The system of claim 1, wherein the power source, the base, and the at least one electrode form an applicator.

3. The system of claim 1, wherein the base and the at least one electrode form an applicator, and wherein the power source is a component of a generator separate from the applicator.

4. The system of claim 1, wherein the at least one electrode comprises a cylindrical electrode.

5. The system of claim 1, wherein the at least one electrode comprises a plurality of electrodes spaced apart from one another a distance of at least 40 mm.

6. The system of claim 1, wherein the at least one electrode comprises a plurality of electrodes extending from the base in a comb configuration.

7. The system of claim 1, wherein the at least one electrode has a hemicylindrical or hemiellipsoid shape.

8. The system of claim 1, wherein the at least one electrode comprises a first electrode and a second electrode, and wherein the first electrode is configured to generate a positive electric field when the second electrode generates a negative electric field.

9. A hand-held applicator for performing irreversible electroporation within a tissue under a skin surface of a patient, the applicator comprising:
a base; and
at least one electrode extending from the base, wherein the at least one electrode is configured to generate an electric field having a maximum field strength equal to a potential difference applied to the tissue divided by a radius of curvature of a surface of the at least one electrode, while providing a treatment field of less than 0.3 times the maximum field strength a distance of 1 mm from the at least one electrode.

10. The applicator of claim 9, wherein the at least one electrode comprises a first electrode and a second electrode, and wherein the applicator further comprises a semiconductive material between the first and second electrodes.

11. The applicator of claim 9, wherein the at least one electrode comprises a first electrode and a second electrode, and wherein the applicator further comprises a semiconductive material including a polymer and conductive particles, between the first and second electrodes.

12. The applicator of claim 9, wherein the at least one electrode comprises at least two cylindrical electrodes spaced apart from each other on a surface of the base.

13. The applicator of claim 9, wherein the at least one electrode comprises a first set of electrodes and a second set of electrodes, wherein each of the first set of electrodes and the second set of electrodes includes multiple electrodes in a comb configuration.

14. A method of performing irreversible electroporation within a tissue under a skin surface of a patient, the method comprising:
applying power to at least one electrode in contact with the skin surface of the patient;
generating with the at least one electrode, based on the applied power, an electric field, wherein the electric field is sufficient to porate a cell membrane within the tissue while increasing a temperature of the skin surface less than 3 deg C. during poration; and
applying the electric field to porate the cell membrane in the tissue.

15. The method of claim 14, wherein the applying the power comprises applying less than about 10 Kwatts to the at least one electrode.

16. The method of claim 14, wherein the generating comprises generating a bipolar or biphasic electric field.

17. The method of claim 14, wherein the generating comprises generating a bipolar electric field with a coaxial pulse former.

18. The method of claim 14, wherein the generating comprises generating the electric field during a movement of the electrode across the skin surface.

19. The method of claim 14, wherein the applying the electric field comprises applying the electric field to porate the cell membrane in the tissue without porating any cell membrane more than 1 mm from the at least one electrode.

20. The method of claim 14, wherein the skin surface is not abraded or cut prior to the applying the electric field.
